SEQUENCE LISTING (36 sequences)

SEQ ID NO: 1 - DNA, 20 bp, Artificial Sequence
  Human Oct-3/4 forward primer
cgaccatctgccgctttgag

SEQ ID NO: 2 - DNA, 20 bp, Artificial Sequence
  Human Oct-3/4 reverse primer
ccccctgtcccccattccta

SEQ ID NO: 3 - DNA, 20 bp, Artificial Sequence
  Human Nanog forward primer
tgctattcttcggccagttg

SEQ ID NO: 4 - DNA, 20 bp, Artificial Sequence
  Human Nanog reverse primer
tgcctcacacggagactgtc

SEQ ID NO: 5 - DNA, 29 bp, Artificial Sequence
  Rex-1 forward primer
tgcatgcattccggttacatgtgtgtaac

SEQ ID NO: 6 - DNA, 27 bp, Artificial Sequence
  Rex-1 reverse primer
ttagagctcggctaggagttcagctcc

SEQ ID NO: 7 - DNA, 24 bp, Artificial Sequence
  Human NeuroD forward primer
aagccatgaacgcagaggaggact

SEQ ID NO: 8 - DNA, 20 bp, Artificial Sequence
  Human NeuroD reverse primer
agctgtccatggtaccgtaa

SEQ ID NO: 9 - DNA, 19 bp, Artificial Sequence
  Human keratin forward primer
gcgggaaagagcctgcagt

SEQ ID NO: 10 - DNA, 25 bp, Artificial Sequence
  Human keratin reverse primer
aaagcacagatcttcgggagctacc

SEQ ID NO: 11 - DNA, 25 bp, Artificial Sequence
  Human T(Brachyury) forward primer
aggaaatcatctcaggaggaagggc

SEQ ID NO: 12 - DNA, 20 bp, Artificial Sequence
  Human T(Brachyury) reverse primer
ttccccgttcacgtacttcc

SEQ ID NO: 13 - DNA, 20 bp, Artificial Sequence
  Human alpha-FP forward primer
agaacctgtcacaagctgtg

SEQ ID NO: 14 - DNA, 20 bp, Artificial Sequence
  Human alpha-FP reverse primer
gacagcaagctgaggatgtc

SEQ ID NO: 15 - DNA, 20 bp, Artificial Sequence
  Human GATA4 forward primer
tccctcttccctcctcaaat

SEQ ID NO: 16 - DNA, 20 bp, Artificial Sequence
  Human GATA4 reverse primer
tcagcgtgtaaaggcatctg

SEQ ID NO: 17 - DNA, 26 bp, Artificial Sequence
  Human GAPDH forward primer
tgaaggtcggagtcaacggatttggt

SEQ ID NO: 18 - DNA, 24 bp, Artificial Sequence
  Human GAPDH reverse primer
catgtgggccatgaggtccaccac

SEQ ID NO: 19 - DNA, 20 bp, Artificial Sequence
  Mouse Wnt1 forward primer
tgcacctgcgactaccggcg

SEQ ID NO: 20 - DNA, 20 bp, Artificial Sequence
  Mouse Wnt1 reverse primer
ctggctccctctgctcttga

SEQ ID NO: 21 - DNA, 20 bp, Artificial Sequence
  Mouse Wnt2 forward primer
gtgcgcggggtcttcgggct

SEQ ID NO: 22 - DNA, 20 bp, Artificial Sequence
  Mouse Wnt2 reverse primer
aaggccgattcccgactact

SEQ ID NO: 23 - DNA, 20 bp, Artificial Sequence
  Mouse Wnt3 forward primer
gccgacttcggggtgctggt

SEQ ID NO: 24 - DNA, 20 bp, Artificial Sequence
  Mouse Wnt3 reverse primer
cttgaagagcgcgtacttag

SEQ ID NO: 25 - DNA, 20 bp, Artificial Sequence
  Mouse Wnt3a forward primer
tggctcctctcggatacctc

SEQ ID NO: 26 - DNA, 20 bp, Artificial Sequence
  Mouse Wnt3a reverse primer
aaagctactccagcggaggc

SEQ ID NO: 27 - DNA, 20 bp, Artificial Sequence
  Mouse Wnt4 forward primer
tccctgcgactcctcgtctt

SEQ ID NO: 28 - DNA, 20 bp, Artificial Sequence
  Mouse Wnt4 reverse primer
gtcactgcaaaggccacacc

SEQ ID NO: 29 - DNA, 20 bp, Artificial Sequence
  Mouse Wnt5a forward primer
ctggaggtgccatgtcttcc

SEQ ID NO: 30 - DNA, 20 bp, Artificial Sequence
  Mouse Wnt5a reverse primer
tcggctgcctatttgcatca

SEQ ID NO: 31 - DNA, 20 bp, Artificial Sequence
  Mouse Wnt7a forward primer
tctcagcctgggcatagtct

SEQ ID NO: 32 - DNA, 20 bp, Artificial Sequence
  Mouse Wnt7a reverse primer
acagtcgctcaggttgccct

SEQ ID NO: 33 - DNA, 20 bp, Artificial Sequence
  Mouse Wnt10b forward primer
ctcgcgggtctcctgttctt

SEQ ID NO: 34 - DNA, 20 bp, Artificial Sequence
  Mouse Wnt10b reverse primer
agtccatcacgatgccagtg

SEQ ID NO: 35 - DNA, 20 bp, Artificial Sequence
  Mouse beta-actin forward primer
atggagaaaatctggcacca

SEQ ID NO: 36 - DNA, 20 bp, Artificial Sequence
  Mouse beta-actin reverse primer
agcatgcatgaccccagcag

(19) United States
(12) Patent Application Publication
(10) Pub. No.: US 2006/0030042 A1
(43) Pub. Date: Feb. 9, 2006

(54) Maintenance of embryonic stem cells by the GSK-3 inhibitor 6-bromoindirubin-3'-oxime

(76) Inventors: Ali Brivanlou, New York, NY (US); Noboru Sato, Durham, NC (US); Laurent Meijer, Roscoff (FR)

Correspondence Address: JONES DAY, 222 EAST 41ST ST, NEW YORK, NY, 10017, US

(21) Appl. No.: 11/018,784

(22) Filed: Dec. 20, 2004

Related U.S. Application Data
(60) Provisional application No. 60/531,250, filed on Dec. 19, 2003.

(51) Int. Cl. C12N 5/08

(52) U.S. Cl. 435/366

(57) ABSTRACT

The present invention relates to methods for maintaining the undifferentiated state of embryonic stem cells without the use of a feeder layer by activating the Wnt signal transduction pathway or by inhibiting glycogen synthase kinase-3 activity by contacting the cell with, inter alia, 6-bromoindirubin-3′-oxime. The present invention also relates to embryonic stem cell lines and cells derived therefrom that have been isolated and cultured in the absence of a feeder layer.

The present application claims benefit under 35 U.S.C. § 119(e) of U.S. Provisional Application Ser. No. 60/531,250 filed Dec. 19, 2003, which is incorporated herein by reference in its entirety.

### 1. INTRODUCTION

The present invention relates to methods for maintaining the undifferentiated state of embryonic stem cells without the use of a feeder layer by activating the Wnt signal transduction pathway or by inhibiting glycogen synthase kinase-3 activity by contacting the cell with, inter alia, 6-bromoindirubin-3′-oxime. The present invention also relates to embryonic stem cell lines and cells derived therefrom that have been isolated and cultured in the absence of a feeder layer.

### 2. BACKGROUND OF THE INVENTION

During early embryogenesis, the first critical fate decision occurs at the blastocyst stage where the embryo is divided into two major lineages, the inner cell mass (ICM) that generates all three germ layer tissues (pluripotency) and trophoblasts supporting embryonic growth (1) (2). Embryonic stem cells are self-renewing cell lines initially derived from the ICM of mouse blastocysts, and proven to inherit pluripotency(1,3, 4). The recent derivation of HESCs opened the door to investigations on the molecular pathways that regulate early human embryogenesis and to generation of human-derived tissues for cell replacement therapy (5, 6). Despite their substantial impact on developmental biology and tissue engineering, little is known about the signaling pathways that govern the unique ESC properties. Leukemia inhibitory factor (LIF)/Stat3 signaling is the only known pathway involved in self-renewal of MESCs. However, loss of function experiments indicate that this pathway is dispensable before gastrulation, suggesting the existence of other signaling cascades essential for ESCs self-renewal(1). We have begun the molecular dissection of signaling pathways functioning in MESCs and HESCs, by taking genetic and biochemical approaches. Large scale gene expression profiling of HESCs reveals that components of several signal transduction pathways are transcriptionally enriched in the undifferentiated state, allowing a prioritization of the pathways to be studied (Sato et al. 2003). Among them, main components of the canonical Wnt pathway are detected in undifferentiated HESCs (Sato et al. 2003). We therefore began to evaluate the potential involvement of the Wnt pathway in the self-renewal ability of MESCs and HESCs. Recently, we have discovered a novel GSK-3 inhibitor, 6-bromoindirubin-3′-oxime, derived from the Mollusk Tyrian purple (Meijer L. et al. 2003). This pharmacological inhibitor inactivates GSK-3 function at much lower concentrations than LiCl, thus facilitating efficient Wnt activation. We have taken advantage of this unique GSK-3 inhibitor to modulate the Wnt pathway for dissecting the molecular mechanism that regulates self-renewal in MESCs and HESCs.

Citation or identification of any reference in Section 2 or in any other section of this application shall not be construed as an admission that such reference is available as prior art to the present invention.

### 3. SUMMARY OF THE INVENTION

The present invention is directed to a method of maintaining the undifferentiated state of an embryonic stem cell, preferably a mammalian embryonic stem cell, more preferably a mouse embryonic stem cell (MESC), or a primate stem cell, even more preferably, a human embryonic stem cell (HESC), said method comprising contacting the stem cell in vitro with a molecule that activates Wnt signal transduction such that the cell divides but does not differentiate. In an aspect of this embodiment, the contacting step is in the absence of a cultured cell feeder layer. In another aspect of this embodiment, the molecule can be the Wnt protein or a fragment thereof, which fragment binds the frizzled receptor. In another aspect the molecule is an agonist of frizzled receptor activation. In another embodiment, the present invention is directed to a method of maintaining the undifferentiated state of an embryonic stem cell, preferably a mammalian embryonic stem cell, more preferably a mouse embryonic stem cell (MESC) or, even more preferably, a human embryonic stem cell (HESC), said method comprising contacting the stem cell in vitro with a molecule that antagonizes glycogen synthase kinase-3 (GSK-3) activity such that the cell divides but does not differentiate. In one aspect of this embodiment the contacting step is in the absence of a feeder layer. In a preferred aspect of this embodiment, the molecule is LiCl. In a more preferred aspect, the molecule is a 6-bromoindirubin, most preferably, 6-bromoindirubin-3′-oxime or a derivative thereof.

The present invention is based, in part, upon the inventors' observation that activation of the Wnt signal transduction pathway or inhibition of glycogen synthase kinase-3 (GSK-3) phosphorylation activity will maintain an embryonic stem cell in its undifferentiated state (i.e., retains totipotency or, at least, pluripotency) without the use of a layer of feeder cells. This maintenance is fully reversible such that inhibiting the Wnt signal transduction pathway or promoting the activity of GSK-3 phosphorylation activity after activation or inhibition, respectively, allows the embryonic stem cell to be able to differentiate. Thus, the present invention solves a long-standing problem of maintaining embryonic stem cells in culture in the absence of feeder cells, i.e., culturing the embryonic stem cells such that they do not differentiate, remain pluripotent, and maintain their ability to self-renew, giving rise to additional stem cells, without an underlying feeder cell layer that can be a source of contamination. In particular, embryonic stem cell lines can be derived and maintained completely in the absence of feeder cells (or even other potential sources of contamination, such as, but not limited to, culture medium conditioned by cells from other cell lines, cell extracts, animal sera, etc.). Such embryonic stem cell lines that have not been exposed to such sources of contamination have particular use in developing therapies for use in human subjects.

The present invention is also directed to a method of maintaining the undifferentiated state of an embryonic stem cell, preferably a mammalian embryonic stem cell, more preferably a mouse embryonic stem cell (MESC) or a primate stem cell, or even more preferably, a human embryonic stem cell (HESC), said method comprising contacting said stem cell with 6-bromoindirubin-3′-oxime or a derivative thereof such that the cells divides but does not differentiate. In one aspect of this embodiment the contacting step is in the absence of a feeder layer. In another aspect, the contacting step is in vitro. Exemplary sources of embryonic stem cells include, but are not limited to, bovine, ovine, equine, porcine sources, such as cows, pigs, horses, chickens, etc.

In another embodiment, the present invention is directed to an isolated embryonic stem cell, preferably a mammalian embryonic stem cell, more preferably a mouse embryonic stem cell (MESC) or, even more preferably, a human embryonic stem cell (HESC), in contact with 6-bromoindirubin-3′-oxime or a derivative thereof. In another embodiment, the invention provides an embryonic stem cell that is the progeny of a second embryonic stem cell, preferably a mammalian embryonic stem cell, more preferably a mouse embryonic stem cell (MESC) or, even more preferably, a human embryonic stem cell (HESC), that was previously contacted with 6-bromoindirubin-3′-oxime or a derivative thereof. In particular aspects, the embryonic stem cells are isolated.

The present invention also provides an embryonic stem cell line produced by the process comprising isolating embryonic stem cells from an embryo and culturing the isolated embryonic stem cells in the presence of a molecule that activates Wnt signal transduction such that the isolated embryonic stem cells divide but do not differentiate. In a particular aspect, the steps of isolating and culturing are in the absence of a feeder layer so that the cells of the cell line and their ancestors have not been in contact with heterologous cultutred cells that could potentially contaminate the embryonic cell line. The embryo is preferably a mammalian embryo, more preferably a mouse embryo or, even more preferably, a human embryo. The present invention also provides an embryonic stem cell line produced by the process comprising isolating embryonic stem cells from an embryo and culturing the isolated embryonic stem cells in the presence of a molecule that antagonizes GSK-3 activity such that the isolated embryonic stem cells divide but do not differentiate. In a particular aspect, the steps of isolating and culturing are in the absence of a feeder layer. In preferred aspects, the molecule is 6-bromoindirubin-3′-oxime or a derivative thereof or the molecule is LiCl. The present invention also provides an embryonic stem cell line produced by the process comprising isolating embryonic stem cells from an embryo, preferably a mammalian embryo, more preferably a mouse embryo or, even more preferably, a human embryo, and culturing the isolated cells in the presence of 6-bromoindirubin-3′-oxime or a derivative thereof such that the isolated embryonic stem cells divide but do not differentiate. In one aspect, the culturing step is in the absence of a feeder layer. In alternative embodiments, the embryonic stem cells are isolated from parthenogenic blastocysts.

In yet another embodiment, the present invention provides a method of obtaining an embryonic stem cell line comprising isolating embryonic stem cells, preferably mammalian embryonic stem cells, more preferably mouse embryonic stem cells (MESCs) or, even more preferably, human embryonic stem cells (HESCs), from an embryo and culturing the isolated embryonic stem cells in the presence of a molecule that activates Wnt signal transduction such that the isolated embryonic stem cells divide but do not differentiate, wherein said isolating and culturing is in the absence of a feeder layer. In certain aspects, the molecule is the Wnt protein or a fragment thereof, which fragment binds the frizzled receptor or the molecule is an agonist of frizzled receptor activation. In another aspect, the activation of Wnt signal transduction can be measured by the sustained or increased expression of Oct-3/4, Rex-1 or Nanog. In another embodiment, the invention provides a method of obtaining an embryonic stem cell line comprising isolating embryonic stem cells, preferably mammalian embryonic stem cells, more preferably mouse embryonic stem cells (MESCs) or, even more preferably, human embryonic stem cells (HESCs), from an embryo and culturing the isolated embryonic stem cells in the presence of a molecule that antagonizes GSK-3 activity such that the isolated embryonic stem cells divide but do not differentiate. In one aspect, the isolating and culturing steps are in the absence of a feeder layer. In preferred aspects, the molecule is LiCl or the molecule is 6-bromoindirubin-3′-oxime or a derivative thereof, including but not limited to Me 6-bromoindirubin-3′-oxime. In yet another embodiment, a method of obtaining an embryonic stem cell line is provided, which method comprises isolating embryonic stem cells, preferably mammalian embryonic stem cells, more preferably mouse embryonic stem cells (MESCs) or, even more preferably, human embryonic stem cells (HESCs), from an embryo and culturing the isolated embryonic stem cells in the presence of 6-bromoindirubin-3′-oxime or a derivative thereof such that the embryonic stem cells divide but do not differentiate. In one aspect, the isolating and culturing steps are in the absence of a feeder layer. In alternative embodiments, the embryonic stem cells are isolated from parthenogenic blastocysts.

In other embodiments, the present invention provides a human embryonic cell line, which cell line has not been in contact with a feeder layer and has not been in contact with an exogenous cell extract or a human embryonic cell line, which cell line has not been in contact with a feeder layer and has not been in contact with a recombinant human protein. In yet other embodiments, the present invention provides differentiated cells, including but not limited to neuronal cells, muscle cells, heart cells, skin cells, bone cells, cartilage cells, liver cells, pancreas cells, hematopoietic cells, lung cells, kidney cells, etc., which are obtained from the embryonic stem cells of the present invention. Such cells can be obtained, e.g., according to the methods described in U.S. Pat. Nos. 5,843,780 and 6,200,806 to Thomson.

In other embodiments, the embryonic stem cells of the present invention are isolated and cultured in the absence of exogenous cell extract or serum. In another embodiment, the embryonic stem cells of the present invention have not been exposed to culture medium that has been conditioned by cells from other cell lines. In yet other embodiments, the embryonic stem cells of the present invention are recombinant embryonic stem cells, which preferably express a prophylactic or therapeutic protein, either overexpressing an endogenous protein or expressing a heterologous protein.

In other specific embodiments of the invention, 6-bromoindirubin-3′-oxime or a derivative thereof may be used to treat, ameliorate, prevent or manage diseases and disorders caused by or associated with a decrease in Wnt pathway signaling and/or with activation of GSK-3 activity. In particular, 6-bromoindirubin-3′-oxime can be used to treat, ameliorate, prevent or manage diseases and disorders involving aberrant cell proliferation and/or differentiation.

### DEFINITIONS

As used herein, a “therapeutically effective amount” refers to that amount of the therapeutic agent sufficient to treat or manage a disease or disorder associated with aberrant Wnt signaling or GSK-3 activation. A therapeutically effective amount may refer to the amount of therapeutic agent sufficient to delay or minimize the onset of the disease or disorder. A therapeutically effective amount may also refer to the amount of the therapeutic agent that provides a therapeutic benefit in the treatment or management of the disease or disorder. Further, a therapeutically effective amount with respect to a therapeutic agent of the invention means that amount of therapeutic agent alone, or in combination with other therapies, that provides a therapeutic benefit in the treatment or management of such diseases or disorders.

As used herein, a “prophylactically effective amount” refers to that amount of the prophylactic agent sufficient to result in the prevention of a disease or disorder associated with aberrant Wnt signaling or GSK-3 activation; including prevention of the onset, recurrence, worsening or spread of such disease or disorder. A prophylactically effective amount with respect to a prophylactic agent of the invention means that amount of prophylactic agent alone, or in combination with other agents, that provides a prophylactic benefit in the prevention of such disease or disorder.

As used herein, the terms “therapeutic agent” and “therapeutic agents” refer to any agent(s) that can be used in the prevention, treatment, or management of a disease or disorder associated with aberrant Wnt signaling or GSK-3 activation.

As used herein, the terms “therapies” and “therapy” can refer to any protocol(s), method(s) and or agent(s) that can be used in the prevention, treatment, or management of diseases or disorders associated with aberrant Wnt signaling or GSK-3 activation.

As used herein, the terms “prophylactic agent” and “prophylactic agents” refer to any agent(s) that can be used in the prevention of the recurrence or spread of a disease or disorder associated with aberrant Wnt signaling or GSK-3 activation.

As used herein, a “therapeutic protocol” refers to a regimen of timing and dosing of one or more therapeutic agents.

As used herein, a “prophylactic protocol” refers to a regimen of timing and dosing of one or more prophylactic agents.

A used herein, a “protocol” includes dosing schedules and dosing regimens.

As used herein, “in combination” refers to the use of more than one prophylactic and/or therapeutic agents.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the term “adjunctive” is used interchangeably with “in combination” or “combinatorial.” Such terms are also used where two or more therapeutic or prophylactic agents affect the treatment or prevention of the same disease.

As used herein, the terms “manage”, “managing” and “management” refer to the beneficial effects that a subject derives from a prophylactic or therapeutic agent, which does not result in a cure of the disease. In certain embodiments, a subject is administered one or more prophylactic or therapeutic agents to “manage” a disease so as to prevent the progression or worsening of the disease.

As used herein, the terms “prevent”, “preventing” and “prevention” refer to the prevention of the recurrence, spread, worsening or onset of a disease in a subject resulting from the administration of a prophylactic or therapeutic agent.

As used herein, the terms “treat”, “treating” and “treatment” refer to the eradication, reduction or amerlioration or symptons of a disease or disorder.

### 4. BRIEF DESCRIPTION OF THE FIGURES

FIGS. 1a-e LIF-induced Stat3 activation is not sufficient to maintain the undifferentiated state of HESCs. (a) H1 or BGN1 cells grown in the feeder free system with conditioned medium (CM). (b) HESCs cultured in non-CM or LIF-containing non-CM demonstrate flattened differentiated morphology. Insets show high power fields. (c) BGN1 cells grown in different conditions were stained with an Oct-3/4-specific antibody. Right panels depict the phase contrast image. (d) Intensity of the Oct-3/4 expression level in each condition was quantified by the image analyzing system. (e) H1, BGN1 or CJ7 (MESCs) cells were treated with LIF, and analyzed by Western analysis using antibodies to Stat3, phosphor (Tyr705)-Stat3, ERK1/2 or phosphorylated ERK1/2. Scale bars: (left panels in a, all panels in b) 300 μm; (right panels in a, insets in b, all panels in c) 100 μm.

FIGS. 2a-e MESCs and HESCs can transduce Wnt signaling by treatment with a GSK-3 inhibitor, 6-bromoindirubin-3′-oxime. (a) Chemical structure of 6-bromoindirubin-3′-oxime and a 6-bromoindirubin-3′-oxime-derivative (Me 6-bromoindirubin-3′-oxime). (b) CJ7 cells were transfected with reporter constructs (TopFlash or FopFlash), treated with 6-bromoindirubin-3′-oxime or Me 6-bromoindirubin-3′-oxime and subjected to the luciferase reporter assay. (c) H1 cells grown in different conditions (conditioned medium; CM, non-CM, 6-bromoindirubin-3′-oxime 2 μM or 5 μM) were incubated with the β-catenin-specific antibody and subjected to confocal microscopic image analysis. Note that HESCs cultured with 6-bromoindirubin-3′-oxime demonstrate nuclear accumulation of β-catenin. Cells were counter stained with DAPI (bottom right panel). Scale bars: (left panels) 20 μm; (right panels) 10 μm. (d) CJ-TY cells were grown in the presence or absence of LIF, and YFP expression was quantitatively determined by the image analyzing system (e) Intensity of YFP expression. Scale bars: 100 μm.

FIGS. 3A-3B Wnt signal activation by 6-bromoindirubin-3′-oxime up-regulates Rex-1 reporter activity. (A) CJ7 cells were transfected with the Rex-1 reporter plasmid and effector constructs, treated with test compounds and evaluated by the luciferase reporter assay. (B) CJRex-Y cells were cultured in different conditions (LIF, non-LIF, Me 6-bromoindirubin-3′-oxime 2 μM or 6-bromoindirubin-3′-oxime 2 μM). Note that cells incubated with 6-bromoindirubin-3′-oxime exhibit a robust YFP expression level and, to some extent, more compact colonies (inset) than those of LIF-treated cells (bottom right panel). Scale bars: 100 μm.

FIGS. 4a-d Activation of Wnt through 6-bromoindirubin-3′-oxime maintains HESCs in the undifferentiated state. (a) CJ7 cells treated with test compounds were examined by the Rex-1 reporter assay. (b) BGN2 cells cultured in non-CM with Wnt3a protein for 5 d were subjected to immunocytochemistry. Scale bars: (all panels except bottom right panel 100 μm (bottom right panel) 50 μm. (c) H1 or BGN1 cells cultured in Me6-bromoindirubin-3′-oxime (2 μM), 6-bromoindirubin-3′-oxime (2 μM) or LiCl (5 or 10 mM)-containing non-conditioned medium (CM) were examined by immunocytochemistry. Note that large majority of cells treated with 6-bromoindirubin-3′-oxime express a strong level of Oct-3/4 with compact undifferentiated morphology. (d) H1 cells cultured in different conditions for 7 d were evaluated by Northern analysis using a human Oct-3/4 or Nanog-specific probe. The similar result was obtained by using BGN1 cells (data not shown). Scale bars: 100 μm.

FIGS. 5a-e Wnt activation in HESCs by 6-bromoindirubin-3′-oxime preserves normal multi-differentiation potentials. (a) H1 cells cultured in conditioned medium (CM), non-CM, Me 6-bromoindirubin-3′-oxime, or 6-bromoindirubin-3′-oxime were induced to form embryoid bodies (EBs). The number of EBs in each condition was counted in triplicate. (b) EBs derived from CM or 6-bromoindirubin-3′-oxime-treated cells were analyzed by RT-PCR. (c) EBs (top left panel, EBs derived from 6-bromoindirubin-3′-oxime-treated cells) were further differentiated and evaluated by immunocytochemistry. Cells stained with GFAP or α-FP antibody are counter stained with DAPI. Scale bars: (all panels except top right and bottom left panels) 100 μm; (top right and bottom left panels) 50 μm. (d) H1 cells were grown in different conditions, and differentiated on PA6 feeder cells. A robust generation of neurons (Tuj-1 positive cells) is constantly observed in 6-bromoindirubin-3′-oxime treated cells. (e) The number of wells containing Tuj-1 positive cells was counted in each group in repeated experiments. Scale bars: (top panel) 300 μm; (bottom panel) 100 μm.

FIG. 6 MESCs maintain pluripotency through 6-bromoindirubin-3′-oxime-mediated Wnt activation. For teratoma formation, MESCs grown in medium containing 6-bromoindirubin-3′-oxime 1 μM were subcutaneously injected into syngenic mice. Teratomas were subjected to hematoxylin and eosin staining for histological examinations. All three germ layer-derived tissues including neuroepithelum (ectoderm, top left panel), cartilage (mesoderm, top right panel), ciliated epithelium (endoderm, bottom left panel) and mucus-producing epithelium (endoderm, bottm right panel) are observed. CJ-GFP cells grown in medium containing 1 μM of 6-bromoindirubin-3′-oxime were microinjected into mouse blastocysts. Embryos at E10.5 were subjected to immunohistochemistry. Representative fluorescent images of injected embryos show colonization of GFP-positive cells in several tissues (top panel; left side: head, bottom panel; left side: dorsal trunk). Scale bars: (top middle & right panels) 100 μm; (bottom left panel) 10 μm; (bottom right panel) 20 μm.

FIG. 7 are photographs of cells showing cyclin D expression evidencing that BIO up-regulates cyclin D1 expression in human embryonic stem cells. BGN2 cells were grown in conditioned medium (CM), non-conditioned medium (non-CM), non-CM with Wnt3a protein (100 ng) or non-CM with 6-bromoindirubin-3′-oxime (1.0 μM) for three days. At the end of the culture period, cells were fixed and incubated with anti-cyclin D1 antibody. Wnt3a or 6-bromoindirubin-3′-oxime treatment up-regulated cyclin D1 expression as compared to cells grown in non-CM or medium containing Me 6-bromoindirubin-3′-oxime (data not shown). Similar results were obtained from BGN1 or H1 cells (data not shown). Scale bars: 50 μm.

FIG. 8 is a gel demonstrating that MEFs express multiple Wnt genes. Total RNA was extracted from MEFs and reverse transcribed to generate cDNA. One μl of cDNA was PCR amplified with each Wnt gene-specific primers. Wnt2, Wnt4 and Wnt5a transcripts are detected among the genes examined by RT_PCR, suggesting that MEFs secrete multiple Wnt ligands.

FIGS. 9A-E are graphs demonstrating that Wnt3a-conditioned medium maintains Rex-1 transcriptional activity in mouse embryonic stem cells. CJRex-Y cells grown in medium containing LIF (B), medium alone (C), control conditioned medium (D) or Wnt3a-conditioned medium (E) for five days were evaluated by FACS analysis. Parental cells without YFP transgene was used as a negative control (A). A representative result from repeated experiments is demonstrated. Cells grown in Wnt3a-conditioned medium exhibit Rex-1 reporter activity at a level comparable to that of cells grown in the presence of LIF, whereas cells cultured in medium alone or control conditioned medium show a significantly lower level of reporter activity. This result further substantiates a primary role of Wnt signaling in the maintenance of the undifferentiated state.

### 5. DETAILED DESCRIPTION OF THE INVENTION

The present invention is directed to a method of maintaining the undifferentiated state of an embryonic stem cell, preferably a mammalian embryonic stem cell, more preferably a mouse embryonic stem cell (MESC), or a primate stem cell, even more preferably, a human embryonic stem cell (HESC), said method comprising contacting the stem cell in vitro with a molecule that activates Wnt signal transduction such that the cell divides but does not differentiate. In an aspect of this embodiment, the contacting step is in the absence of a cultured cell feeder layer. In another aspect of this embodiment, the molecule can be the Wnt protein or a fragment thereof, which fragment binds the frizzled receptor. In another aspect the molecule is an agonist of frizzled receptor activation. In another embodiment, the present invention is directed to a method of maintaining the undifferentiated state of an embryonic stem cell, preferably a mammalian embryonic stem cell, more preferably a mouse embryonic stem cell (MESC) or, even more preferably, a human embryonic stem cell (HESC), said method comprising contacting the stem cell in vitro with a molecule that antagonizes glycogen synthase kinase-3 (GSK-3) activity such that the cell divides but does not differentiate. In one aspect of this embodiment the contacting step is in the absence of a feeder layer. In a preferred aspect of this embodiment, the molecule is LiCl. In a more preferred aspect, the molecule is a 6-bromoindirubin, most preferably, 6-bromoindirubin-3′-oxime or a derivative thereof. The present invention is also directed to a method of maintaining the undifferentiated state of an embryonic stem cell, preferably a mammalian embryonic stem cell, more preferably a mouse embryonic stem cell (MESC) or a primate stem cell, or even more preferably, a human embryonic stem cell (HESC), said method comprising contacting said stem cell with 6-bromoindirubin-3′-oxime or a derivative thereof such that the cells divides but does not differentiate. In one aspect of this embodiment the contacting step is in the absence of a feeder layer. In another aspect, the contacting step is in vitro.

The present invention is based, in part, upon the inventors' observation that activation of the Wnt signal transduction pathway or inhibition of glycogen synthase kinase-3 (GSK-3) phosphorylation activity will maintain an embryonic stem cell in its undifferentiated state (i.e., retains totipotency or, at least, pluripotency) without the use of a layer of feeder cells. An embryonic stem cell retains the ability to differentiate into trophoblasts as well as all three germ layers (endoderm, ectoderm and mesoderm). This maintenance is fully reversible such that inhibiting the Wnt signal transduction pathway or promoting the activity of GSK-3 phosphorylation activity after activation or inhibition, respectively, allows the embryonic stem cell to be able to differentiate. Thus, the present invention solves a long-standing problem of maintaining embryonic stem cells in culture in the absence of feeder cells, i.e., culturing the embryonic stem cells such that they do not differentiate, remain pluripotent, and maintain their ability to self-renew, giving rise to additional stem cells, without an underlying feeder cell layer that can be a source of contamination. In particular, embryonic stem cell lines can be derived and maintained completely in the absence of feeder cells (or even other potential sources of contamination, such as, but not limited to, cell extracts, animal sera, etc.). Such embryonic stem cell lines that have not been exposed to such sources of contamination have particular use in developing therapies for use in human subjects.

Any methods know in the art for culturing the embryonic stem cells can be employed in the present invention, including those described in Section 6, infra. Further, the cells are contaced with a molecule that activates Wnt signal transduction or with a molecule that inactivates GSK-3 activity using methods that all commonly known in the art, including those described in Section 6, infra. For example, the molecule is added to the culture medium containing the cells.

Activators of Wnt signal transduction pathway are also known in the art, e.g., Wnt proteins and fragments that bind frizzled, and frizzled receptor agonists, such as activating antibodies to frizzled. Inhibitors of GSK-3 activity are also known in the art and include, but are not limited to, anti-GSK-3 antibodies and intrabodies. Such molecules are preferably non-toxic to the embryonic stem cells. An exemplary and preferred inhibitor of GSK-3 is a 6-bromoindirubin. A most preferred inhibitor is 6-bromoindirubin-3′-oxime or a derivative thereof. Effective concentrations of 6-bromoindirubin-3′-oxime in the culture medium for maintaining the undifferentiated state are about 0.001 μM to about 100 μM, preferably about 0.1 to about 10 μM, and most preferably 1 μM. The maintenance of the undifferentiated states can be correlated with expression of transcription factors Oct-3/4, Rex-1 and Nanog, in which sustained or increased expression of these factors indicates that the undifferentiated state is being maintained. Further, assays detecting the expression of Oct-3/4, Rex-1 or Nanog can be used to determine effective or optimal concentrations of an activator of the Wnt signal transduction pathway, or inhibitors of GSK-3 activity or of a 6-bromoindirubin.

In another embodiment, the present invention is directed to an isolated embryonic stem cell, preferably a mammalian embryonic stem cell, more preferably a mouse embryonic stem cell (MESC) or a primate embryonic stem cell, or even more preferably, a human embryonic stem cell (HESC), in contact with 6-bromoindirubin-3′-oxime or a derivative thereof. In particular, such embryonic stem cells are derived and maintained completely in the absence of feeder cells (or even other potential sources of contamination, such as, but not limited to, cell extracts, animal sera, etc.), and, thus, such embryonic stem cells have not been exposed to such sources of contamination. For example, the embryonic stem cells can be obtaining and culturing cells from the inner cell mass of a blastocyst, culturing in the presence of an activiator of Wnt signal transduction or an inhibitor of GSK-3 activity in the absence of feeder cells or heterologous proteins and identifying colonies of stem cells. See also U.S. Pat. Nos. 5,843,780 and 6,200,806 to Thomson for illustrative methods for isolating and culturing stem cells.

In another embodiment, the invention provides an embryonic stem cell that is the progeny of a second embryonic stem cell, preferably a mammalian embryonic stem cell, more preferably a mouse embryonic stem cell (MESC) or, even more preferably, a human embryonic stem cell (HESC), that was previously contacted with 6bromoindirubin-3′-oxime or a derivative thereof. In particular aspects, the embryonic stem cells are isolated.

The present invention also provides an embryonic stem cell line produced by the process comprising isolating embryonic stem cells from an embryo and culturing the isolated embryonic stem cells in the presence of a molecule that activates Wnt signal transduction such that the isolated embryonic stem cells divide but do not differentiate. In a particular aspect, the steps of isolating and culturing are in the absence of a feeder layer so that the cells of the cell line and their ancestors have not been in contact with heterologous cultutred cells that could potentially contaminate the embryonic cell line. The embryo is preferably a mammalian embryo, more preferably a mouse embryo or, even more preferably, a human embryo. The present invention also provides an embryonic stem cell line produced by the process comprising isolating embryonic stem cells from an embryo and culturing the isolated embryonic stem cells in the presence of a molecule that antagonizes GSK-3 activity such that the isolated embryonic stem cells divide but do not differentiate. In a particular aspect, the steps of isolating and culturing are in the absence of a feeder layer. In preferred aspects, the molecule is 6-bromoindirubin-3′-oxime or a derivative thereof or the molecule is LiCl. The present invention also provides an embryonic stem cell line produced by the process comprising isolating embryonic stem cells from an embryo, preferably a mammalian embryo, more preferably a mouse embryo or, even more preferably, a human embryo, and culturing the isolated cells in the presence of 6-bromoindirubin-3′-oxime or a derivative thereof such that the isolated embryonic stem cells divide but do not differentiate. In one aspect, the culturing step is in the absence of a feeder layer. In alternative embodiments, the embryonic stem cells are isolated from parthenogenic blastocysts.

In yet another embodiment, the present invention provides a method of obtaining an embryonic stem cell line comprising isolating embryonic stem cells, preferably mammalian embryonic stem cells, more preferably mouse embryonic stem cells (MESCs) or, even more preferably, human embryonic stem cells (HESCs), from an embryo and culturing the isolated embryonic stem cells in the presence of a molecule that activates Wnt signal transduction such that the isolated embryonic stem cells divide but do not differentiate, wherein said isolating and culturing is in the absence of a feeder layer. In certain aspects, the molecule is the Wnt protein or a fragment thereof, which fragment binds the frizzled receptor or the molecule is an agonist of frizzled receptor activation. In another aspect, the activation of Wnt signal transduction can be measured by the sustained or increased expression of Oct-3/4, Rex- or Nanog. In another embodiment, the invention provides a method of obtaining an embryonic stem cell line comprising isolating embryonic stem cells, preferably mammalian embryonic stem cells, more preferably mouse embryonic stem cells (MESCs) or, even more preferably, human embryonic stem cells (HESCs), from an embryo and culturing the isolated embryonic stem cells in the presence of a molecule that antagonizes GSK-3 activity such that the isolated embryonic stem cells divide but do not differentiate. In one aspect, the isolating and culturing steps are in the absence of a feeder layer. In preferred aspects, the molecule is LiCl or the molecule is 6-bromoindirubin-3′-oxime or a derivative thereof, including but not limited to Me 6-bromoindirubin-3′-oxime. In yet another embodiment, a method of obtaining an embryonic stem cell line is provided, which method comprises isolating embryonic stem cells, preferably mammalian embryonic stem cells, more preferably mouse embryonic stem cells (MESCs) or, even more preferably, human embryonic stem cells (HESCs), from an embryo and culturing the isolated embryonic stem cells in the presence of 6-bromoindirubin-3′-oxime or a derivative thereof such that the embryonic stem cells divide but do not differentiate. In one aspect, the isolating and culturing steps are in the absence of a feeder layer. In alternative embodiments, the embryonic stem cells are isolated from parthenogenic blastocysts.

In other embodiments, the present invention provides a human embryonic cell line, which cell line has not been in contact with a feeder layer and/or has not been in contact with an exogenous cell extract, or a human embryonic cell line, which cell line has not been in contact with a feeder layer and/or has not been in contact with a recombinant human protein. In yet other embodiments, the present invention provides differentiated cells, including but not limited to neuronal cells, muscle cells, heart cells, skin cells, bone cells, cartilage cells, liver cells, pancreas cells, hematopoietic cells, lung cells, kidney cells, etc., which are obtained from the embryonic stem cells of the present invention, as well as tissues produced from these differentiated cells. Such cells can be obtained, e.g., according to the methods described in U.S. Pat. Nos. 5,843,780 and 6,200,806 to Thomson.

In other embodiments, the embryonic stem cells of the present invention are isolated and cultured in the absence of exogenous cell extract or serum. In yet other embodiments, the embryonic stem cells of the present invention are recombinant embryonic stem cells, which preferably express a prophylactic or therapeutic protein, either overexpressing an endogenous protein or expressing a heterologous protein. Further, the stem cells of the present invention as well as progeny cells thereof can be frozen and stored.

### THERAPEUTIC USES OF 6-BROMOINDIRUBIN-3′-OXIME

In other specific embodiments of the invention, 6-bromoindirubin-3′-oxime may be used to treat, ameliorate, prevent or manage diseases and disorders caused by or associated with a decrease in Wnt pathway signaling and/or with activation of GSK-3 activity. In particular, 6-bromoindirubin-3′-oxime can be used to treat, ameliorate, prevent or manage diseases and disorders involving aberrant cell proliferation and/or differentiation.

In specific embodiments, 6-bromoindirubin-3′-oxime can be used to treat, ameliorate, prevent or manage hyperproliferative cell disease, particularly cancer. Cancers and related disorders that can be treated or prevented by methods of the present invention include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, therapy by administration of 6-bromoindirubin-3′-oxime is combined with the administration of one or more therapies such as, but not limited to, chemotherapies, radiation therapies, hormonal therapies, and/or biological therapies/immunotherapies.

In a specific embodiment, the methods of the invention encompass the administration of 6-bromoindirubin-3′-oxime in combination with one or more angiogenesis inhibitors such as but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Additional examples of anti-cancer agents that can be used in the various embodiments of the invention, include, but are not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflomithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, fmasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. Preferred additional anti-cancer drugs are 5-fluorouracil and leucovorin.

The invention also encompasses administration of 6-bromoindirubin-3′-oxime in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (56^{th} ed., 2002).

The invention provides methods of treatment (and prophylaxis) by administration to a subject of an effective amount of 6-bromoindirubin-3′-oxime (the “Therapeutic”). The subject is preferably an animal, including but not limited to animals such as cows, pigs, horses, chickens, cats, dogs, etc., and is preferably a mammal, and most preferably human. In a specific embodiment, a non-human mammal is the subject.

Various delivery systems are known and can be used to administer a Therapeutic of the invention, e.g., encapsulation in liposomes, microparticles, microcapsules, receptor-mediated endocytosis (see, e.g., Wu and Wu, 1987, J. Biol. Chem. 262:4429-4432), etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the Therapeutic locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the Therapeutic can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

The present invention also provides pharmaceutical compositions comprising 6-bromoindirubin-3′-oxime for use in the methods of the invention. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutic of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention that will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The following series of examples are presented by way of illustration and not by way of limitation on the scope of the present invention.

### 6. EXAMPLES

Introduction

Human and mouse embryonic stem cells (HESCs and MESCs, respectively) self-renew indefinitely while maintaining the ability to generate all three germ layer derivatives. Despite their substantial impact on developmental biology and tissue replacement therapy, the molecular mechanism underlying HESCs properties is poorly understood. Here we show that activation of the canonical Wnt pathway is sufficient for the maintenance of self-renewal of both ESCs. Although Stat3 signaling is involved in MESCs self-renewal, stimulation of this pathway fails to support self-renewal of HESCs lines. Instead, we find that Wnt activation by a pharmacological GSK-3-specific inhibitor, 6-bromoindirubin-3′-oxime, maintains the undifferentiated phenotype in both ESCs, and sustains expression of pluripotent state-specific transcription factors, Oct-3/4, Rex-1 and Nanog. Supporting this, Wnt signaling is endogenously activated in undifferentiated MESCs, and downregulated upon differentiation. Moreover, 6-bromoindirubin-3′-oxime-mediated Wnt activation is functionally reversible as withdrawal of the compound leads to normal multi-differentiation programs in both ESCs. The results demonstrate that use of Wnt pathway activators and GSK-3-specific inhibitors like 6-bromoindirubin-3′-oxime are beneficial to practical applications in regenerative medicine.

Materials and Methods

Chemicals. 6-bromoindirubin-3′-oxime and its kinase inactive analogue, 1-methyl-6-bromoindirubin-3′-oxime (Me 6-bromoindirubin-3′-oxime) were prepared as described in detail elsewhere¹⁰. LiCl was purchased from Sigma.

Cell culture. Human embryonic stem cells (HESCs) lines were provided by WiCell Research Institute (H1 line)⁵ and BresaGen Inc. (BGN1 and BGN2 lines). H1 cells were cultivated on irradiated mouse embryonic fibroblasts (MEFs) in medium consisting of 80% DMEM/F12 medium, 20% knockout serum replacement (KSR), 1 mM L-glutamine, 1% non-essential amino acids, 0.1 mM β-mercaptoethanol, and 4 ng/ml basic FGF (all from Invitrogen). BGN1 and BGN2 cells were originally cultured in essentially the same medium as for H1 but with 15% FBS (HyClone) and an initial concentration of 5% KSR instead of 20% KSR, then, gradually adapted to a higher concentration of KSR to completely shift to the same fully defined medium for H1 cells. Subsequently, HESCs were cultured on Matrigel (BD biosciences) in medium conditioned by MEFs, and passaged several times until colonies became free from contaminating MEFs as described before⁹. Normal karyotype was confirmed by the standard method (data not shown). For in vitro experiments, HESCs were cultured for 3 to 7 d in conditioned medium, or non-conditioned medium in the presence or absence of mouse or human LIF (Chemicon International), or recombinant mouse Wnt3a protein (100 ng/ml, R&D systems) added to fresh medium everyday, or compounds as indicated in the Result section.

Mouse embryonic stem cells (MESCs) including CJ7 (provided by W. Mark, Memorial Sloan-Kettering Institute) or E14 (provided by C. Yang, The Rockefeller University) line were maintained on MEFs in mouse ES cell medium containing knockout Dulbecco's minimal essential medium supplemented with 15% FBS, 100 mM MEM nonessential amino acids, 0.55 mM 2-mercaptoethanol, and 1 mM L-glutamine (all from Invitrogen). To remove MEFs, cells were harvested by trypsinization, plated on 10 cm dishes for 30 min, and non-adherent cells consisting mainly of ES cells were replated on gelatin or Matrigel-coated dishes (1000 cells/cm²) and grown in mouse ES medium supplemented with 1400 U/ml LIF.

Blastocyst injection. We cultured CJ-GFP cells at a low density (500 cells/cm²) on gelatin-coated 10 cm dishes in medium containing 6-bromoindirubin-3′-oxime 1 μM for 5 d. 6-bromoindirubin-3′-oxime-treated cells (approximately 10 to 15 cells per blastocyst) were microinjected into each blastocyst and transferred into surrogate mice in the C57B1/6 background. Mid-gestation embryos were recovered and subjected to tissue sectioning. Chimerism of live offspring was determined by evaluation of their mixed coat color.

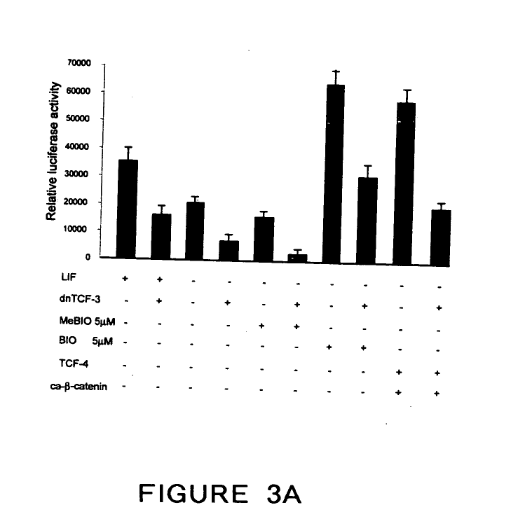

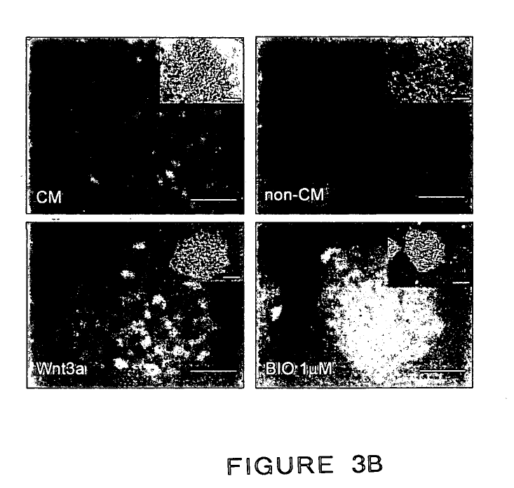

Teratoma formation. We grew CJ-GFP cells at a low density (500 cells/cm²) on gelatin-coated 10 cm dishes in medium containing 6-bromoindirubin-3′-oxime 1 μM for 7 d, then passaged cells at the same density and cultured under the same condition another 5 d to further enforce differentiation of cells. Despite this extensive differentiation culture protocol without LIF, at the end of the culture period, most of 6-bromoindirubin-3′-oxime-treated cells still formed round tight colonies like cells grown in medium containing LIF as seen in FIG. 3b, whereas medium alone or Me 6-bromoindirubin-3′-oxime-treated cells demonstrated large flat differentiated morphology (data not shown). 6-bromoindirubin-3′-oxime-treated cells (approximately 5×10⁶ cells/mouse) were subcutaneously injected into the left flank of syngenic 129 background mice. After three to five weeks, the developed teratoma (approximately 20 mm in diameter) was excised, fixed in 4% paraformaldehyde and subjected to hematoxylin and eosin staining for histological examinations.

All animal studies were approved by the Animal Care and Use Committee of The Rockefeller University.

Immunofluorescence. Cells were fixed in 4% paraformaldehyde for 20 min at room temperature and incubated overnight at 4° C. with primary antibodies against Oct-4 (BD Biosciences), β-catenin (BD Biosciences), Tuj-1 (BAbCO), cytokeratin (Sigma), glial fibrillary acidic protein, GFAP, (Dako), smooth muscle actin (Research Diagnostics Inc), α-fetoprotein (α-FP, Cell Sciences) and Tromal (Developmental Studies Hybridoma Bank). For nuclear localization analysis, the fixed samples were subjected to fluorescent digital confocal imaging analysis using a Zeiss LSM 510 confocal microscope (Carl Zeiss). For mouse embryonic tissue sections, mid gestation embryos were fixed in 4% paraformaldehyde followed by paraffin embedding and tissue sectioning. After deparaffinization, tissue sections were incubated with a GFP-specific antibody (Molecular Probe) at 4° C. overnight. Antigens were localized by using goat anti-mouse IgG conjugated to Cy3 or goat anti-rabbit IgG conjugated to Cy2 (Zymed laboratories). For the quantitative image analysis, fluorescent images from triplicate samples were taken by the Discoveryl system (Universal Imaging Corporation). The fluorescent objects were selected by the threshold function and evaluated in five regions of each well by quantification of pixel intensities and the object size by using MetaMorph software (Universal Imaging Corporation).

Northern analysis. Total RNA was isolated from cells by using Qiashredder and RNAeasy mini kit (Qiagen). The extracted RNA sample was quantified by UV spectrophotometer, and qualified by the RNA Nano Lab chip (Agilent Technologies). Ten μg of total RNA was electrophoresed on 1% agarose/formaldehyde gel and transferred onto a nylon membrane (Stratagene). Probes specific for human Oct-4 and human Nanog were prepared by RT-PCR using gene specific primer pairs (shown below) and the template cDNA generated from undifferentiated H1 cells, and radio-labeled with ³²P-dCTP by Prime-it probe labeling kit (Stratagene). The membrane was hybridized with the labeled probe using Perfecthybri (Sigma) and subjected to detection by Phosphor Imager (Amersham Biosciences).

(SEQ ID NO:1)

Human Oct-3/4 forward primer:
5′-CGACCATCTGCCGCTTTGAG-3′

(SEQ ID NO:2)

Human Oct-3/4 reverse primer:
5′-CCCCCTGTCCCCCATTCCTA-3′

(SEQ ID NO:3)

Human Nanog forward primer:
5′-TGCCTCACACGGAGACTGTC-3′

(SEQ ID NO:4)

Human Nanog reverse primer:
5′-TGCTATTCTTCGGCCAGTTG-3′

Western analysis. Total protein was extracted with lysis buffer (50 mM Tris/150 mM NaCl/0.1% Triton X-100/0.1 mM DTT and proteinase inhibitors). Protein concentrations were determined by BCA Protein Assay kit (Pierce). 50 μg of protein was separated by 10% SDS/PAGE and transferred onto a nylon membrane (BioRad, Hercules, Calif.). The membrane was incubated with antibodies to β-catenin, Stat3, phosphorylated Stat3 (tyr705), ERK1/2, phosphorylated ERK1/2 (Thr202/204) (Cell Signaling Technology), followed by incubation with peroxidase-conjugated goat anti-mouse IgG or goat anti-rabbit IgG (Jackson immunoResearch), and developed with ECL reagent (Amersham Biosciences).

Embryoid body (EB) formation. HESCs were harvested by using dispase (Invitrogen), plated on non-tissue culture treated dishes (approximately 10⁷ cells/10 cm dish), and grown in non-conditioned medium for 7 d as described before (Sato et al., 2003, Molecular signature of human embryonic stem cells and its comparison with the mouse. Dev Biol 260,404-413). The number of EBs was determined by counting EBs in 20 different fields at a low magnification (10×) using an Axiovert microscope (Zeiss). Experiments were repeated at least three times, and the average number as well as standard deviation were calculated. For the detection of differentiated derivatives by immunocytochemistry, EBs (day 7) were plated on collagen-coated 12-well plates to allow them to adhere, grown in DMEM containing 10% FBS to induce further differentiation for 7 d and fixed in 4% paraformaldehyde followed by immunostaining as shown below.

Differentiation of ES cells into neurons. The PA6 stromal cell line (RIKEN) was used for the co-culture system (Kawasaki et al., 2002, Generation of dopaminergic neurons and pigmented epithelia from primate ES cells by stromal cell-derived inducing activity. Proc Natl Acad Sci USA 99, 1580-1585). H1 cell colonies grown on Matrigel under different conditions for 7 d were harvested and cultured (100˜200 cells/clump, approximately 5 clumps/well of a 12-well plate) on PA6 stromal cells in 90% Knockout-Dulecco's modified Eagle's medium, 10% KSR, 1 mM L-glutamine, 1% non-essential amino acids, 0.1 mM β-mercaptoethanol for 3 weeks as previously described (Sato et al., 2003, Molecular signature of human embryonic stem cells and its comparison with the mouse. Dev Biol 260, 404-413). At the end of the culture period, cells were stained with a neuron-specific antibody, Tuj-1, as shown below. The number of wells containing Tuj-1 positive neurons was determined in each condition in repeated experiments.

Plasmid construction. pTopFlash and pFopFlash were provided by H. Clevers (Netherlands Institute for Developmental Biology) (Korinek et al., 1997, Constitutive transcriptional activation by a beta-catenin-Tcf complex in APC−/− colon carcinoma. Science 275, 1784-1787). To generate a reporter construct (pTY) carrying the mutant form of YFP (Venus), a gift from A. Miyawaki (Brain Science Institute, RIKEN) Nagai et al., 2002, A variant of yellow fluorescent protein with fast and efficient maturation for cell-biological applications. Nat Biotechnol 20, 87-90), a small fragment containing the TCF binding sites and the cFos promoter in pTopFlash was excised by XbaI digestion, and cloned into pcDNA3-Venus in which Venus was introduced into the multiple cloning site between BamHI and EcoRI of the pcDNA3 vector (Invitrogen) whose CMV promoter was eliminated by BglII and HindIII digestion. The Rex-1 promoter region was PCR amplified from the mouse Rex-1 genomic fragment, a gift from L. Gudas (Weill Medical College) (Hosler ET AL., 1989, Expression of REX-1, a gene containing zinc finger motifs, is rapidly reduced by retinoic acid in F9 teratocarcinoma cells. Mol Cell Biol 9, 5623-5629), with specific primer pairs as shown below and subcloned into the pGL2-Basic vector (Promega). To generate the Rex-1-Venus reporter construct, the Rex-1 promoter region was cloned into pcDNA3-Venus. The pCAG-HygEGFP construct was assembled by insertion of a SalI-KpnI fragment containing the CAG promoter region of the pCAG vector, a gift from J. Miyazaki (University of Osaka) (Niwa et al., 1991, Efficient selection for high-expression transfectants with a novel eukaryotic vector. Gene 108, 193-199), into pIRES.hrGFP (Stratagene) in which the CMV promoter was removed by NsiI and NotI digestion followed by insertion of the HygEGFP fragment from the pHygEGFP vector (BD Clontech) into the multiple cloning site.

(SEQ ID NO:5)

Rex-1 forward primer:
5′-TGCATGCATTCCGGTTACATGTGTGTAAC-3′

(SEQ ID NO:6)

Rex-1 reverse primer:
5′-TTAGAGCTCGGCTAGGAGTTCAGCTCC-3′

Generation of stable mouse ES lines. CJ7 ES cells were transfected with pRex-1-Venus, pTY or pCAG-HygEGFP by using Lipofectamine 2000 (Invitrogen) followed by G418 (Invitrogen) selection at 200 μg/ml (for CJRex-Y or CJ-TY) or hygromycine (Invitrogen) selection at 600 μg/ml (for CJ-GFP). Two weeks after the drug selection, a number of single colonies were picked up, expanded and used for the further analyses.

RT-PCR. Two μg of total RNA extracted from EBs (day 7) or mouse embryonic fibroblasts was reverse-transcribed using ThermoScript RT-PCR system (Invitrogen) according to the manufacturer's protocol. One μl of cDNA sample was PCR amplified with each gene-specific primers (shown below) using optimized PCR cycles to obtain amplified reactions in a linear range.

Human NeuroD forward primer (Henderson et al., 2002, Preimplantation human embryos and embryonic stem cells show comparable expression of stage-specific embryonic antigens. Stem Cells 20, 329-337): 5′-AAGCCATGAACGCAGAGGAGGACT-3′ (SEQ ID NO:7) Human NeuroD reverse primer: 5′-AGCTGTCCATGGTACCGTAA-3′ (SEQ ID NO:8) Human keratin forward primer: 5′-AGGAAATCATCTCAGGAGGAAGGGC-3′ (SEQ ID NO:9) Human keratin reverse primer: 5′-AAAGCACAGATCTTCGGGAGCTACC-3′ (SEQ ID NO: 10) Human T (Brachyury) forward primer: 5′-GCGGGAAAGAGCCTGCAGTA-3′ (SEQ ID NO:11). Human T reverse primer: TTCCCCGTTCACGTACTTCC-3′ (SEQ ID NO:12) Human α-FP forward primer: 5′-AGAACCTGTCACAAGCTGTG-3′ (SEQ ID NO:13) Human α-FP reverse primer: 5′-GACAGCAAGCTGAGGATGTC-3′ (SEQ ID NO:14) Human GATA4 forward primer: 5′-TCCCTCTTCCCTCCTCAAAT-3′ (SEQ ID NO:15) Human GATA4 reverse primer: 5′-TCAGCGTGTAAAGGCATCTG-3′ (SEQ ID NO:16) Human GAPDH forward primer: 5′-TGAAGGTCGGAGTCAACGGATTTGGT-3′ (SEQ ID NO:17) Human GAPDH reverse primer: 5′-CATGTGGGCCATGAGGTCCACCAC-3′ (SEQ ID NO:18) Mouse Wnt1 forward primer: 5′-TGCACCTGCGACTACCGGCG-3′ (SEQ ID NO:19) Mouse Wnt1 reverse primer: 5′-GTGCGCGGGGTCTTCGGGCT-3′ (SEQ ID NO:20) Mouse Wnt2 forward primer: 5′-CTGGCTCCCTCTGCTCTTGA-3′ (SEQ ID NO:21) Mouse Wnt2 reverse primer: 5′-AAGGCCGATTCCCGACTACT-3′ (SEQ ID NO:22) Mouse Wnt3 forward primer: 5′-GCCGACTTCGGGGTGCTGGT-3′ (SEQ ID NO:23) Mouse Wnt3 reverse primer: 5′-CTTGAAGAGCGCGTACTTAG-3′ (SEQ ID NO:24) Mouse Wnt3a forward primer: 5′-TGGCTCCTCTCGGATACCTC-3′ (SEQ ID NO:25) Mouse Wnt3a reverse primer: 5′-AAAGCTACTCCAGCGGAGGC-3′ (SEQ ID NO:26) Mouse Wnt4 forward primer: 5′-TCCCTGCGACTCCTCGTCTT-3′ (SEQ ID NO:27) Mouse Wnt4 reverse primer: 5′-GTCACTGCAAAGGCCACACC-3′ (SEQ ID NO:28) Mouse Wnt5a forward primer: 5′-CTGGAGGTGCCATGTCTTCC-3′ (SEQ ID NO:29) Mouse Wnt5a reverse primer: 5′-TCGGCTGCCTATTTGCATCA-3′ (SEQ ID NO:30) Mouse Wnt7a forward primer: 5′-TCTCAGCCTGGGCATAGTCT-3′ (SEQ ID NO:31) Mouse Wnt7a reverse primer: 5′-ACAGTCGCTCAGGTTGCCCT-3′ (SEQ ID NO:32) Mouse Wnt10b forward primer: 5′-CTCGCGGGTCTCCTGTTCTT-3′ (SEQ ID NO:33) Mouse Wnt10b reverse primer: 5′-AGCATGCATGACCCCAGCAG-3′ (SEQ ID NO:34) Mouse β-actin forward primer: 5′-ATGGAGAAAATCTGGCACCA-3′ (SEQ ID NO:35) Mouse β-actin reverse primer: 5′-AGTCCATCACGATGCCAGTG-3′ (SEQ ID NO:36)

Luciferase assay. Cells (MESCs; 5000 cells/cm² in the 24-well plate or HESCs; approximately 50 cells/clump, 100 clumps/cm² in the 24-well plate) were transfected with the firefly or Renilla reporter plasmid (MESCs; 100 ng or 10 ng per well, respectively, or HESCs; 500 ng or 20 ng per well, respectively) and specific constructs including dnXTCF-3 (a gift from A. Vonica, The Rockefeller University) (Molenaar et al., 1996, XTcf-3 transcription factor mediates beta-catenin-induced axis formation in Xenopus embryos. Cell 86, 391-399; Vonica et al., 2002, Zygotic Wnt activity is required for Brachyury expression in the early Xenopus laevis embryo. Dev Biol 250, 112-127), human TCF-4 and ca-β-catenin (gifts from H. Clevers) (Korinek et al., 1997, Constitutive transcriptional activation by a beta-catenin-Tcf complex in APC−/− colon carcinoma. Science 275, 1784-1787), (MESCs; 100 ng per well except pdnXTCF-3 used at 300 ng per well or HESCs; 500 ng per well) in triplicate by using Lipofectamine 2000 (Invitrogen) according to the manufacturer's protocol. Test compounds were added 24 hrs after transfection. Following incubation for 24 hrs, cells were harvested, and analyzed by the dual luciferase reporter assay system (Promega) using Lumat (Berthold Technologies). Each value was standardized by Renilla luciferase activity.

1FACS analysis of MESCs. We cultured CJRex-Y cells at a low density (500 cells/cm²) on gelatin-coated 10 cm dishes in mouse ES cell medium alone, medium containing LIF, medium conditioned from wild type L cells or medium conditioned from Wnt3a-L cells (ATCC) for 5 days. The Wnt3a conditioned medium was prepared according to the provider's protocol. Cells were harvested, resuspended in mouse ES medium and subjected to FACS analysis using FACSVantage SE system (BD Bioscienses).

Immunofluorescence. HESCs were grown in conditioned medium (CM), non-CM, non-CM containing Me 6-bromoindirubin-3′-oxime (1.0 μM), non-CM containing 6-bromoindirubin-3′-oxime (1.0 μM) or non-CM containing recombinant mouse Wnt3a protein (100 ng/ml, R&D systems) for 5 d. Cells were fixed in 4% paraformaldehyde for 20 min at room temperature and incubated overnight at 4° C. with primary antibodies against cyclin D1 (Santa Cruz Technology, Santa Cruz, Calif.). Antigens were localized by using goat anti-rabbit IgG conjugated to Cy3 (Zymed laboratories).

Results

LIF-Induced Stat3 Activation does not Sustain the Undifferentiated State in HESCs

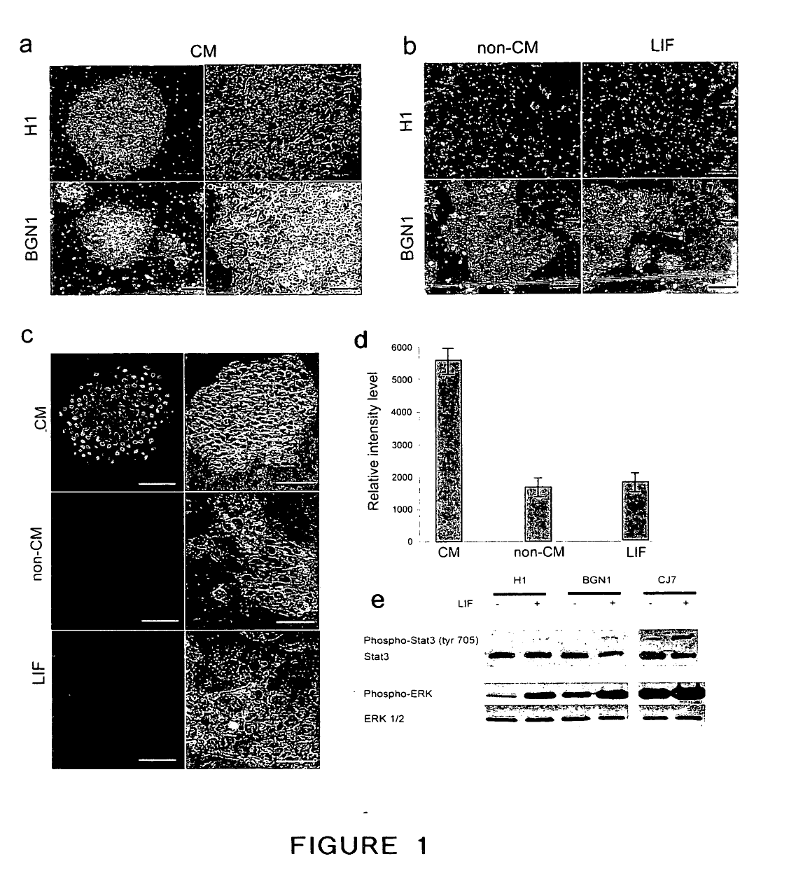

Although the LIF/Stat3 pathway is currently the only pathway known to be involved in the self-renewal of MESCs¹, its role has not been clearly demonstrated in HESCs^{5,6}. To evaluate this, we used a feeder-free culture system in which HESCs are physically free from mouse embryonic fibroblasts (MEFs), thereby making their differentiation state merely dependent on culture medium¹¹. We used three independent HESCs lines, H1 (WiCell)⁵, BGN1 and BGN2 (BresaGen), that are successfully maintained in the undifferentiated state with medium conditioned from MEFs. The normal karyotype was confirmed after several passages (data not shown). Undifferentiated H1 and BGN1 cells showed typical compact morphology with a high nuclear-cytoplasmic ratio comparable to that seen in HESCs grown on MEFs⁵ (FIG. 1a). As early as 24 hrs after replacing conditioned medium with non-conditioned medium, HESCs started to flatten and reached a completely differentiated cell morphology after 5 to 7 d incubation (FIG. 1b). To confirm and quantify the differentiation process, we monitored the expression of Oct-3/4, a key transcription factor for pluripotency restricted to the ICM of blastocysts¹²⁻¹⁴ by immunocytochemical analysis. HESCs grown in conditioned medium showed unambiguous nuclear Oct-3/4 staining in each single cell at 7 d in culture, whereas a marked reduction of Oct-3/4 expression was observed in flattened differentiated cells cultured in non-conditioned medium for 7 d (FIG. 1c). This differentiation program was not prevented by addition of LIF even at higher concentrations (2,000 to 3,000 U/ml), suggesting that LIF alone is not sufficient to maintain HESCs undifferentiated (FIG. 1b,c). Quantitative evaluation of Oct-3/4 expression levels by image analysis confirmed these observations (FIG. 1d). Human-derived LIF or a combination of IL6 and soluble IL6 receptor which activate the Stat3 pathway also failed to prevent the differentiation (data not shown). To evaluate whether HESCs are capable of responding to a LIF signal, we carried out Western analysis to determine the phosphorylated (Tyr705) Stat3 protein level that represents the activation status of the Stat3 signaling pathway¹⁵. As shown in FIG. 1e, H1 and BGN1 cells treated with LIF for 20 min showed a weak increase in Tyr705-phosphorylated Stat3 that was not further enhanced in different time points (data not shown), whereas a sharp activation of the ERK pathway was evident upon LIF stimulation in both HESCs lines and MESCs. This contrasts with MESCs showing a marked increase in Tyr705-phosphorylated Stat3 upon LIF treatment, as previously reported¹⁵. These results revealed that, although the Stat3 signaling pathway can be stimulated by LIF in HESCs, the level of activation is far less than ones in the mouse and it does not affect self-renewal in HESCs. Since it could be argued that in human, another Stat might be involved, we also tested the phosphorylation status of Stat1 and Stat5. None of these two Stats showed any sign of activation in HESCs and MESCs (D. Besser, NS and AHB, unpublished observation), eliminating Stat signaling as causal to self-renewal of HESCs. We therefore began to investigate the contribution of other pathways to ESCs self-renewal. Toward this end, we took advantage of both global expression screens using microarrays for MESCs and HESCs^{9,16}, and testing the biochemical state of components of the main pathways¹⁷. Main signal transducers of the canonical Wnt pathway were detected in undifferentiated HESCs in our array experiments (see Table 1 below)⁹. This result prompted us to begin our evaluation with the Wnt pathway.

TABLE 1

| Probe ID | GenBank | Gene |
|---|---|---|
| 219683_at | NM_017412.1 | Homo sapiens frizzled (Drosophila) homolog 3 (FZD3) |
| 206136_at | NM_003468.1 | Homo sapiens frizzled (Drosophila) homolog 5 (FZD5)* |
| 203987_at | NM_003506.1 | Homo sapiens frizzled (Drosophila) homolog 6 (FZD6) |
| 203706_s_at | NM_003507.1 | Homo sapiens frizzled (Drosophila) homolog 7 (FZD7) |
| 219764_at | NM_007197.1 | Homo sapiens frizzled (Drosophila) homolog 10 (FZD10) |
| 34697_at | AF074264 | Homo sapiens LDL receptor-related protein 6 (LRP6) |
| 203230_at | AF006011.1 | Homo sapiens dishevelled 1 (DVL1) |
| 201908_at | NM_004423.2 | Homo sapiens dishevelled 3 (DVL3) |
| 632_at | L40027 | Homo sapiens glycogen synthase kinase 3 α |
| 209945_s_at | BC000251.1 | Homo sapiens glycogen synthase kinase 3 β |
| 219889_at | NM_005479.1 | Homo sapiens frequently rearranged in advanced T-cell |
| | | lymphomas (FRAT1) |
| 209864_at | AB045118.1 | Homo sapiens FRAT2* |
| 201533_at | NM_001904.1 | Homo sapiens catenin (cadherin-associated protein), beta 1 |
| | | (88 kD) |
| | | (CTNNB1) |
| 221016_s_at | NM_031283.1 | Homo sapiens HMG-box transcription factor TCF-3 (TCF-3) |
| 203753_at | NM_003199.1 | Homo sapiens transcription factor 4 (TCF4) |

Components of the Wnt signaling pathway called ‘Present’ in undifferentiated HESCs by gene chip analysis. RNA samples from undifferentiated and differentiated H1 cells were evaluated by using Affymetrix U133A chips followed by statistical analysis 1. A total of 9626 genes were called ‘Present’ in the undifferentiated state among all transcripts (22200) on the U133A chip. Wnt pathway components were selected from the ‘Present’ genes and shown in the table.
*Genes enriched in the undifferentiated state as compared to differentiated HESCs.

MESCs and HESCs can Transduce Wnt Signaling

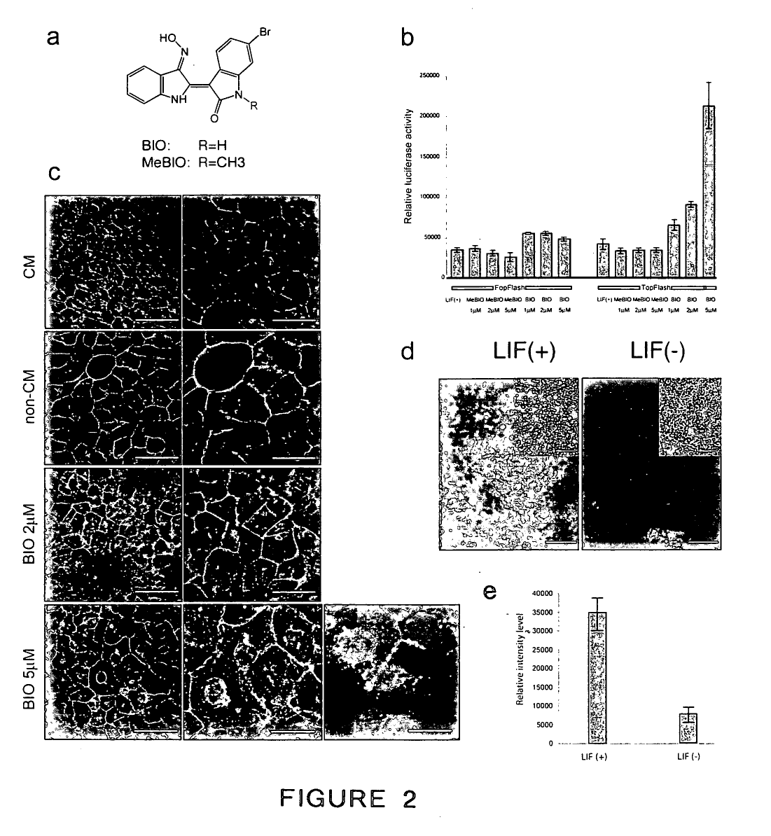

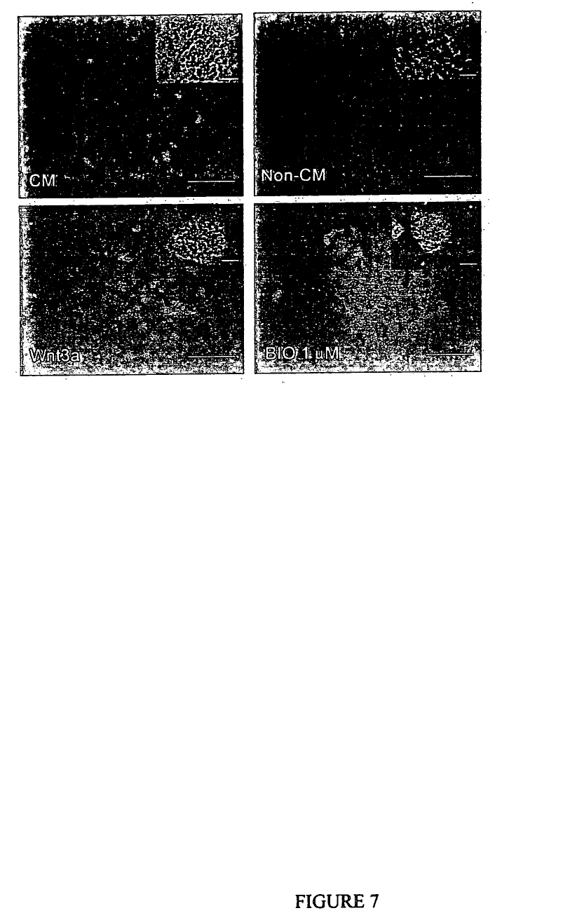

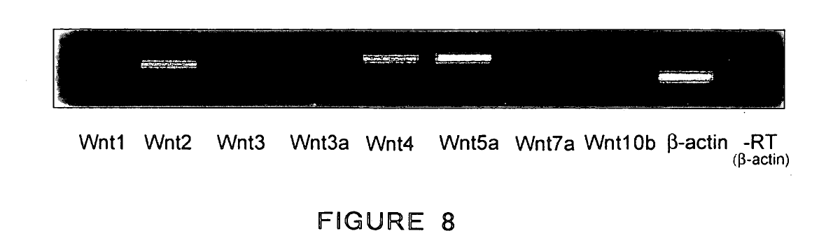

The canonical Wnt signal occurs through binding of the Wnt protein to the Frizzled receptor at cell surface. It is followed by inactivation of GSK-3, leading to accumulation of β-catenin in the nucleus that activates the transcription of Wnt target genes in collaboration with TCFs^{18,19}. Alternatively, Wnt signaling can be activated by direct, intracellular inhibition of the GSK-3 function using specific inhibitors²⁰. We have recently discovered that 6-bromoindirubins, initially derived from Tyrian purple, were rather selective and potent inhibitors of GSK-3¹⁰. Among indirubins, 6-bromoindirubin-3′-oxime, and its kinase-inactive analogue, 1-methyl-6-bromoindirubin-3′-oxime (Me 6-bromoindirubin-3′-oxime) (FIG. 2a), are particularly convenient tools to modulate GSK-3 activity¹⁰. We first determined the activation of the Wnt signaling pathway by 6-bromoindirubin-3′-oxime using 293 human kidney epithelial cells evaluated by a luciferase reporter system in which the promoter module contained TCF binding sites (TopFlash) or non-responsive mutated binding sites (FopFlash)²¹. 6-bromoindirubin-3′-oxime, but not Me 6-bromoindirubin-3′-oxime, robustly upregulated the reporter activity at micromolar concentrations, far below the concentrations required for LiCl-mediated activation, indicating efficient activation of the canonical Wnt pathway by 6-bromoindirubin-3′-oxime (data not shown). Based on this evidence, we decided to use 6-bromoindirubin-3′-oxime as a positive regulator of Wnt signaling in the subsequent experiments. First we examined whether mouse and human ESCs were capable of transducing Wnt signaling under the influence of 6-bromoindirubin-3′-oxime. CJ7 cells (MESCs) treated with 6-bromoindirubin-3′-oxime demonstrated a remarkable increase in TopFlash reporter activity in a dose-dependent manner, whereas Me 6-bromoindirubin-3′-oxime-treated cells did not show any change in activity (FIG. 2b). As expected, no substantial change in FopFlash reporter activity was observed under similar conditions (FIG. 2b). Similar results were obtained using E14 cells, a MESC line in the 129 background (data not shown). We then evaluated expression of β-catenin at the cellular level as a read-out of the activation status of the canonical Wnt pathway in HESCs. 6-bromoindirubin-3′-oxime-treated HESCs showed nuclear accumulation of β-catenin (FIG. 2c) as compared to non-conditioned medium treated cells, whereas Me 6-bromoindirubin-3′-oxime-treated cells did not show obvious difference (data not shown), indicating activated transduction of the canonical Wnt pathway in HESCs by 6-bromoindirubin-3′-oxime. This result was further supported by the observation that cyclin D1, one of the Wnt-target genes²², was upregulated in HESCs treated with 6-bromoindirubin-3′-oxime (FIG. 7). Since MESCs and HESCs are known to be kept undifferentiated in the presence of MEFs, and MEFs express multiple Wnt ligands (FIG. 8), these results raise an intriguing possibility that Wnt proteins secreted from MEFs might activate Wnt signaling in both ESCs in the undifferentiated state.

Wnt Signaling is Activated in Undifferentiated ESCs

To monitor Wnt activity in MESCs for a longer period, we generated a reporter MESCs line (CJ-TY) in which a modified version of the yellow fluorescent protein (YFP)²³ was regulated by the TopFlash promoter module (pTY). Similar to the luciferase reporter data (FIG. 2b), no appreciable difference in the YFP expression level between LIF-treated and untreated cells was observed on day 2 (data not shown). After 5 d of incubation, however, the reporter cells treated with LIF still maintained a strong level of promoter activity with undifferentiated morphology, whereas LIF-untreated cells showed large differentiated cell morphology with a notable decrease in YFP expression (FIG. 2d,e), suggesting downregulation of Wnt activity upon differentiation. This data indicates that Wnt signaling is endogenously active in undifferentiated MESCs.

Activation of Wnt Induces Rex-1 Expression in MESCs

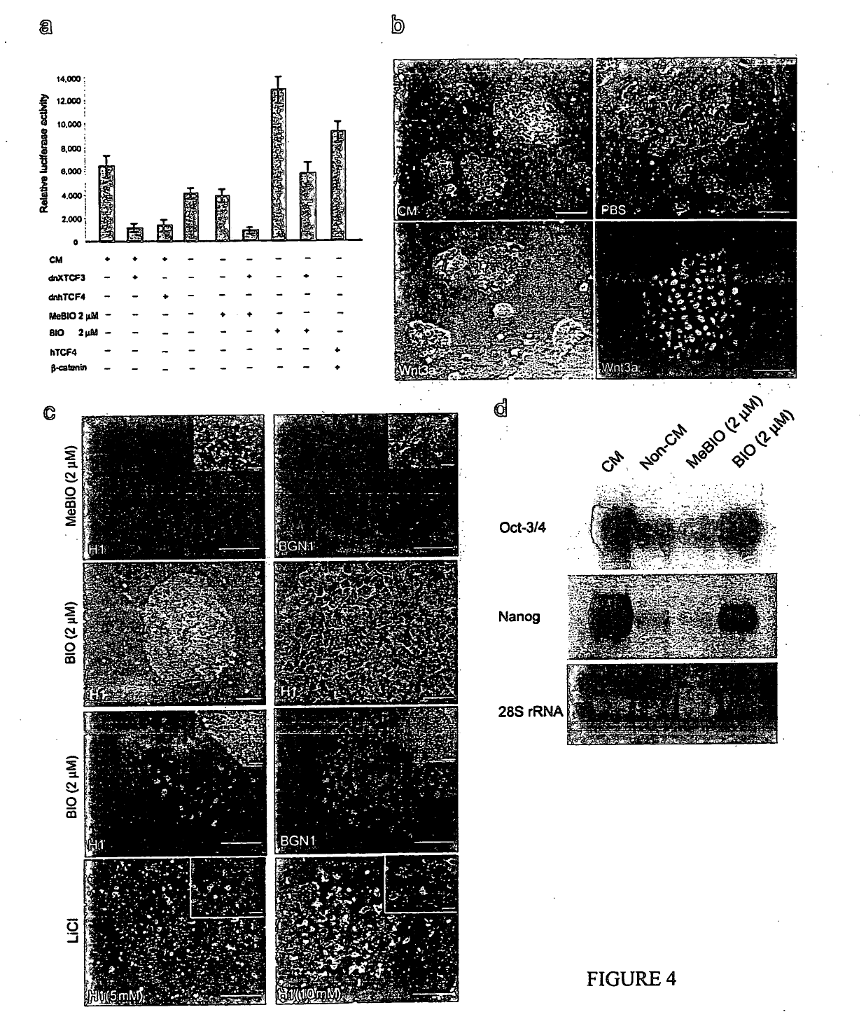

On the basis of these observations, we reasoned that active Wnt signaling might be instrumental in maintaining the molecular machinery responsible for the undifferentiated state. Accordingly loss of Wnt activity might trigger deactivation of the machinery thereby allowing initiation of the differentiation program. To test this hypothesis, we monitored the expression of Rex-1, another molecular marker of pluripotency, in MESCs using a luciferase reporter construct in which the luciferase gene was regulated by the Rex-1 minimal enhancer element²⁴. Compared to LIF-treated CJ7 cells, Rex-1 promoter activity showed substantial upregulation in cells exposed to 6-bromoindirubin-3′-oxime, while it was slightly reduced in cells treated with Me 6-bromoindirubin-3′-oxime or grown in the absence of LIF (FIG. 4a). Similar results were obtained with the E14 cell line, whereas P19 embryonal carcinoma cells²⁴ or non-pluripotent stem cell lines including 293, NIH3T3 and mesenchymal stem cells did not show notable Rex-1 transcriptional activity (data not shown). When transfected with the dominant negative TCF-3 construct which specifically blocks downstream of the canonical Wnt signaling^{25,26}, 6-bromoindirubin-3′-oxime-mediated transcriptional activation was largely abolished, confirming that 6-bromoindirubin-3′-oxime functions through the canonical Wnt pathway. Given that GSK3 regulates multiple pathways including insulin and growth factors-mediated cascades besides Wnt signaling, we could not rule out a possibility that other signaling pathways activated by 6-bromoindirubin-3′-oxime might influence the pluripotency in MESCs. To further substantiate the role of Wnt on Rex-1 transcriptional regulation, a constitutively active form of β-catenin and TCF-4, known to assemble a pivotal transcriptional machinery in the canonical Wnt pathway, were co-transfected, and found to efficiently upregulate Rex-1 promoter activity (FIG. 4a).

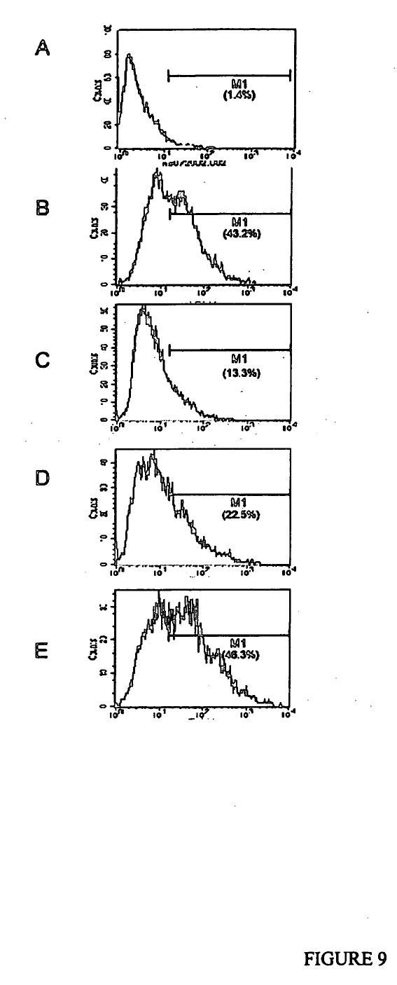

Although Rex-1 reporter activity in LIF-untreated MESCs did not decline drastically at 48 hrs, this time period may be too short to allow cells to differentiate completely as the obvious morphological change was not observed during this period. To monitor Rex-1 transcriptional activity for a longer time at the cellular level, we generated a stable MESCs reporter line (CJRex-Y) expressing a mutant form of YFP regulated by the Rex-1 minimal enhancer²⁴. 6-bromoindirubin-3′-oxime-treated CJRex-Y cells showed strong transcriptional activity as well as colonies, to some extent, more compact than those observed in LIF-treated cells after 5 d of incubation (FIG. 3b), while LIF-untreated or Me 6-bromoindirubin-3′-oxime-treated cells showed substantially lower activity and a flattened cell shape. These data demonstrated that activation of the canonical Wnt pathway by the GSK-3 inhibitor is sufficient to retain the undifferentiated phenotype as well as Rex-1 promoter activity in the absence of LIF. To further determine the role of Wnt signaling in the undifferentiated state, we used Wnt3a conditioned medium instead of 6-bromoindirubin-3′-oxime for stimulation of the Wnt pathway. CJRex-Y cells grown in medium conditioned from L cells that stably express Wnt3a maintained a high level of Rex-1 transcriptional activity comparable to that of LIF-treated cells, whereas medium conditioned from wild type L cells or non LIF-treated cells showed apparently reduced reporter activity as determined quantitatively by FACS analysis (FIG. 9).

Wnt Activation Maintains Undifferentiated Phenotype and Gene Expression in HESCs

We next asked whether the effect of Wnt activation on the undifferentiated state observed in mouse was also conserved in human. To this end, we examined if the undifferentiated state of HESCs in the feeder-free system could be modulated by exogenous activation of the Wnt pathway utilizing GSK-3 inhibitors. Me 6-bromoindirubin-3′-oxime treated H1 and BGN1 cells showed fully flattened morphology after 7 d incubation (FIG. 4c inserts top panel) as observed in non-conditioned medium treated cells (FIG. 1b). Another GSK-3 inhibitor, LiCl, failed to maintain the undifferentiated phenotype at 5 mM and showed substantial toxicity at 10 mM (FIG. 4c bottom panel). In contrast, HESCs treated with 2 μM 6-bromoindirubin-3′-oxime largely retained an undifferentiated morphology (FIG. 4c second top panel) comparable to that of conditioned medium-treated cells (FIG. 1a). This observation was further supported molecularly by monitoring Oct-3/4 expression at the cellular level. Strikingly, sustained Oct-3/4 expression was observed in the majority of HESCs treated with 6-bromoindirubin-3′-oxime, in contrast to the remarkable reduction of Oct-3/4 expression in Me 6-bromoindirubin-3′-oxime-treated differentiated cells (FIG. 4c top panels). Quantitative imaging analysis showed a comparable level of Oct-3/4 expression under 6-bromoindirubin-3′-oxime and conditioned medium treatment conditions (data not shown). We also used recombinant Wnt3a protein to ensure that 6-bromoindirubin-3′-oxime-mediated effect was caused by Wnt activation. Wnt3a-treated cells maintain compact undifferentiated colonies with a high level of Oct-3/4 expression (data not shown) as seen in 6-bromoindirubin-3′-oxime-treated cells, whereas cells cultured in non-CM with PBS (used for reconstitution of Wnt3a protein) showed differentiated morphology with low Oct-3/4 expression (FIG. 4b). To determine whether sustained Oct-3/4 expression is regulated at the transcriptional level, Northern analysis was performed. We found that a substantial level of the Oct-3/4 transcript was maintained in 6-bromoindirubin-3′-oxime-treated HESCs compared to the expression level in conditioned medium-treated cells, while a much reduced level was found in other conditions (FIG. 4d), indicating preservation of the Oct-3/4 transcript level through activation of Wnt. We also used the same Rex-1 reporter assay system for testing HESCs lines as used for MESCs. Since transfection efficiency of H1 cells was extremely low, to obtain reliable reporter activity, BGN1 and BGN2 cells that showed higher transfection efficiency were evaluated. Both lines demonstrated an identical pattern of Rex-1 reporter activity similar to that observed in MESCs (FIG. 4d).

A recent study has revealed a novel homeodomain transcription factor, Nanog, that is both sufficient and required for maintenance of pluripotency in MESCs and mouse epiblasts, independently of Stat3 signaling^{27,28}. Our Northern analysis revealed that a substantial level of Nanog transcripts was preserved in 6-bromoindirubin-3′-oxime-treated HESCs, whereas remarkable reduction was observed in the differentiation conditions (FIG. 4c). Taken together, these results underscore that activation of the canonical Wnt pathway by 6-bromoindirubin-3′-oxime facilitates maintenance of the undifferentiated phenotype as well as positive transcriptional regulation of pluripotent state-specific transcription factors in MESCs and HESCs, implying a conserved role for Wnt signaling in ESCs among mouse and human.

Activation of Wnt Preserves Normal Differentiation Potentials in HESCs

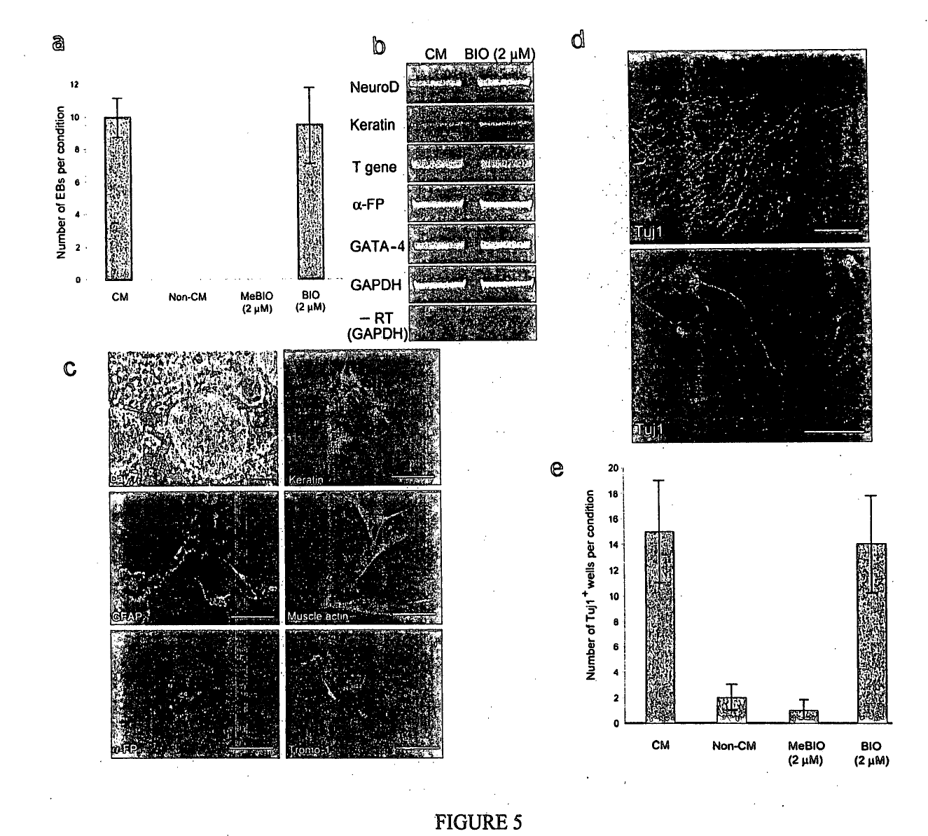

Since one of the unique properties of ESCs is their ability to generate cells with functional diversity, we further explored the differentiation potential of 6-bromoindirubin-3′-oxime-treated ESCs utilizing established ESCs differentiation systems. We first generated embryoid bodies (EBs) consisting of three germ layer derivatives from undifferentiated HESCs^{1,29}. We observed EBs formation from 6-bromoindirubin-3′-oxime-treated H1 cells at a level comparable to that seen with conditioned medium-treated cells, whereas no EB was formed in other conditions (FIG. 5a). Similar results were obtained with BGN1 and BGN2 lines in the same system (data not shown). Lineage-specific marker analysis by RT-PCR exhibited that EBs derived from conditioned medium or 6-bromoindirubin-3′-oxime-treated cells similarly developed into ectoderm (NeuroD and keratin), mesoderm (T gene) and endoderm (α-fetoprotein and GATA4) derivatives (FIG. 5b). To further determine the differentiation phenotype of 6-bromoindirubin-3′-oxime-treated HESCs at the cellular level, EBs were grown under the adherent condition, and evaluated by immunocytochemistry. HESCs initially treated with 6-bromoindirubin-3′-oxime showed a wide variety of morphology and lineage-specific molecule expression including ectoderm (cytokeratin and glial fibrillary acidic protein, GFAP), mesoderm (smooth muscle actin), endoderm (α-fetoprotein) and trophectoderm (tromol) markers (FIG. 5c) at levels comparable to those seen in conditioned medium-treated cells (data not shown).

A growing body of evidence indicates that ESCs can be manipulated to undergo lineage-restricted differentiation programs including neurons by unique culture techniques, subsequently grafted and integrated into host tissues, suggesting possible applications for tissue engineering^{1,7,8}. We therefore tested if 6-bromoindirubin-3′-oxime-treated HESCs retain the capacity to exclusively differentiate into neurons in a stromal co-culture system by which a high level of neurogenesis occurs through stromal-derived factors^{9,30}. We found that 6-bromoindirubin-3′-oxime-treated H1 cells induced a robust neurogenesis on stromal feeders comparable to that seen in conditioned medium-treated cells (FIG. 5d), while much lower efficiency was observed in cells grown in other conditions (FIG. 5e). Similar results were obtained with BGN1 and BGN2 cells (data not shown).

Activation of Wnt Signaling Maintains MESCs in the Pluripotent State

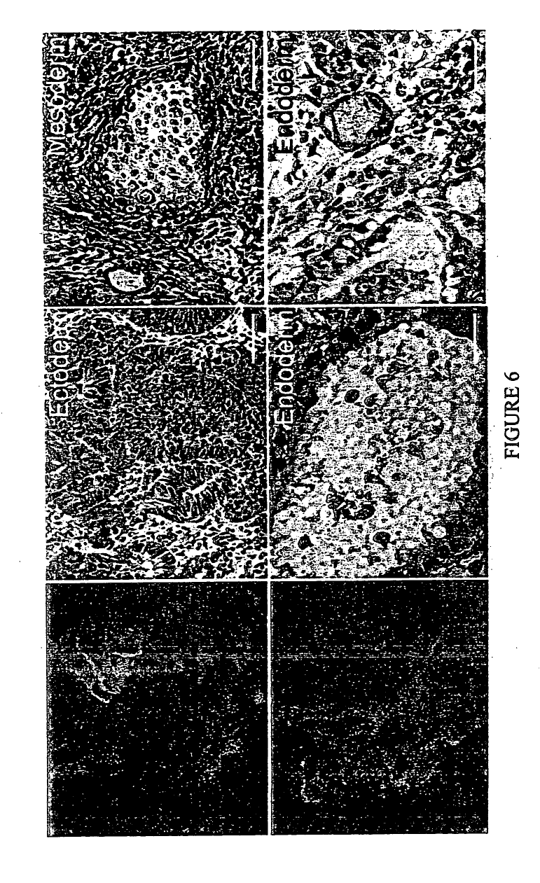

We next addressed whether 6-bromoindirubin-3′-oxime-treated MESCs maintained the ability to form three germ layer derivatives as evaluated by subcutaneous injection of MESCs into syngenic mice. 6-bromoindirubin-3′-oxime-treated MESCs generated teratomas consisting of all three germ layer-derived tissues including neuroepithelium (ectoderm), cartilage (mesoderm) and ciliated epithelium (endoderm) (FIG. 6).

Finally, given that another unique functional property of ESCs is their capacity to synchronize with surrounding embryonic microenvironment, we evaluated if 6-bromoindirubin-3′-oxime-treated MESCs retained the potential to adapt early embryonic differentiation process by chimeric mice generation. We found that 6-bromoindirubin-3′-oxime-treated CJ-GFP cells that constitutively express GFP were incorporated into several embryonic tissues at the mid-gestation stage as determined by immunohistochemistry (FIG. 6). More than 60% of injected embryos contained colonized GFP-positive cells in repeated experiments (11/14; 78%, 8/12; 66%). Our initial assessment of coat-color chimerism of live offspring demonstrated that two of five new-born animals were chimeric.

All together, these results indicate that although activation of Wnt signaling by 6-bromoindirubin-3′-oxime allows ESCs to remain undifferentiated, the precise multi-differentiation program can be properly reactivated upon withdrawal of the exogenous Wnt activating compound, highlighting the preservation of the essential features of ESCs.

Discussion

We demonstrate here that despite the ability of LIF/Stat3 signaling to support self-renewal of MESCs, it failed to prevent the differentiation of three independent HESCs lines, suggesting that this pathway is not essential for self-renewal in HESCs. The LIF/Stat3 pathway, however, has been shown to be dispensable for pregastrulation embryos in mutant mouse studies¹. In addition, an as yet unidentified soluble factor secreted from a differentiated cell line have supported germline transmission of MESCs independently of Stat3 signaling³¹. Our study demonstrates Wnt signaling as a possible common signaling pathway that maintains the undifferentiated state of ESCs of mouse and human origin.

Oct-3/4 and Rex-1 have been studied as representative transcription factors involved in controlling the pluripotent state in MESCs, although little is known about upstream signals that regulate these molecules³². Our results provide a novel insight into regulatory cascades underlying the unique molecular program in ESCs by demonstrating that Wnt signaling can positively regulate transcription of these key molecules in human and mouse. This finding is further expanded by the observation that expression of Nanog, a novel homeoprotein both sufficient and necessary for maintenance of pluripotency^{27,28}, is also transcriptionally sustained by activation of Wnt. Since the preservation of the Oct-3/4 expression level is not sufficient for prevention of differentiation³³, Wnt-dependent ESCs self-renewal might be mediated by transcriptional regulation of Nanog. Further studies are required to identify molecular interactions between these transcription factors and Wnt signaling components in ESCs. Loss of function models of the Wnt pathway have been generated by gene targeting in mice. β-catenin mutant mice are defective in A-P axis formation, but not in maintaining the pluripotent state³⁴. However, since another member of the armadillo family, plakoglobin, redistributes to compensate the adherence function of β-catenin, it might also transduce Wnt signaling in the mutant embryos³⁵. Given that pluripotency is a fundamental biological function in multicellular organisms, it is likely to be evolutionarily secured by multiple genetic backup systems as suggested by expression of several Wnt ligands in preimplantation embryos³⁶.

During early vertebrate embryogenesis, the canonical Wnt pathway has been shown to fulfill early and important embryological functions including its role in the induction of the dorsal organizer (node)¹⁹, via the formation of the Nieuwkoop center¹⁹. It is, therefore, tempting to speculate that in addition to mediating the pathway underlying stemness, the sustained activation of this pathway might lead to the formation of the embryonic node (or organizer) in vitro.

Aberrant activation of Wnt signaling has been implicated in cancer formation in numerous basic and clinical studies^{19,43}. A recent report using mutant MESCs lines in which Wnt signaling was constitutively activated by mutations in adenomatous polyposis coli (APC) or β-catenin, showed sustained undifferentiated morphology and impaired differentiation capacities, reminiscent of uncontrollable immature cell growth in tumors⁴⁴. Although this report is basically consistent with our results, the simple morphological evaluation of the undifferentiated phenotype without monitoring pluripotent-specific molecular markers precludes a precise characterization of the state of stemness in MESCs. More importantly, our system in which Wnt signaling is transiently activated in ESCs by a GSK-3 inhibitor clearly indicates that the retained undifferentiated state is not definitive but readily reversible upon withdrawal of the inhibitor, as illustrated by a series of functional differentiation assays in MESCs and HESCs, highlighting that preserved stemness by Wnt is regulatable. Another recent report has shown that inhibition of Wnt signaling in MESCs accelerates neural differentiation and, conversely, activation of Wnt signaling by Wnt1 overexpression or LiCl treatment resulted in inhibition of neural differentiation⁴⁵. While these results are in line with our observations, since they used a culture condition that specifically induced neural lineages and evaluated progenies mainly by using neuron-specific markers, influence by modulation of Wnt signaling on other germ layer derivatives has not been addressed. A recent study using another pharmacological GSK-3 inhibitor has shown that P19 embryonal carcinoma cells and MESCs can be differentiated into neurons likely through activation of Wnt⁴⁶, contrasting with our observations and previous reports^{44,45}. Although the reason of the different results is unknown, it might be caused by different culture conditions or uncharacterized functions of the compound that might differently affect ESCs identities.

The finding that the undifferentiated HESCs can be reversibly maintained in an undifferentiated state by the mere addition of a synthetic pharmacological compound might open new avenues in the practical applications of HESCs in regenerative medicine. Since large-scale cultivation of a homogenous population of undifferentiated HESCs would be an inevitable and fundamental first step to provide an unlimited source of tissue transplant, the use of the well-defined stable chemical compound might be suitable to regulate standardized quality of HESCs rather than using feeder cell-derived undefined factor(s) required for the current culture protocol. In addition, applying chemically produced indirubins such as 6-bromoindirubin-3′-oxime might eliminate the requirement for all mouse-derived materials from culture conditions including the derivation process. Moreover, these synthetic chemical compounds may also be tested for the expansion of many types of adult stem or progenitor cell populations as their growth seems to be highly dependent on Wnt signaling^{42,47}.

Finally, we demonstrated Wnt signaling as a common pathway for maintenance of the undifferentiated state in both mouse and human ES cells, while LIF signaling is mainly involved in mouse, providing an example of genetic pathways that are functionally different between the two species.

### REFERENCES

1. Smith, A. G. Embryo-derived stem cells: of mice and men. Annu Rev Cell Dev Biol 17, 435-62 (2001).
2. Rossant, J. Stem cells from the Mammalian blastocyst. Stem Cells 19, 477-82 (2001).
3. Martin, G. R. Isolation of a pluripotent cell line from early mouse embryos cultured in medium conditioned by teratocarcinoma stem cells. Proc Natl Acad Sci USA 78, 7634-8. (1981).
4. Evans, M. J. & Kaufman, M. H. Establishment in culture of pluripotential cells from mouse embryos. Nature 292, 154-6. (1981).
5. Thomson, J. A. et al. Embryonic stem cell lines derived from human blastocysts. Science 282, 1145-7. (1998).
6. Reubinoff, B. E., Pera, M. F., Fong, C. Y., Trounson, A. & Bongso, A. Embryonic stem cell lines from human blastocysts: somatic differentiation in vitro. Nat Biotechnol 18, 399-404 (2000).
7. Hori, Y. et al. Growth inhibitors promote differentiation of insulin-producing tissue from embryonic stem cells. Proc Natl Acad Sci USA 99, 16105-10 (2002).
8. Kim, J. H. et al. Dopamine neurons derived from embryonic stem cells function in an animal model of Parkinson's disease. Nature 418, 50-6 (2002).
9. Sato, N. et al. Molecular signature of human embryonic stem cells and its comparison with the mouse. Dev Biol 260, 404-13 (2003).
10. Meijer, L., Skaltsounis, A-L., Magiatis, P., Polychronopoulos, P., Knockaert, M., Leost, M., P. Ryan, X., Vonica, A., Brivanlou, A., Dajani, R., Crovace, C., Tarricone, C., Musacchio, A., Roe, M., Pearl, L. & Greengard, P. GSK-3 selective inhibitors derived from Tyrian purple indirubins. Chem. Biol. in Press.
11. Xu, C. et al. Feeder-free growth of undifferentiated human embryonic stem cells. Nat Biotechnol 19, 971-4. (2001).
12. Okamoto, K. et al. A novel octamer binding transcription factor is differentially expressed in mouse embryonic cells. Cell 60, 461-72. (1990).
13. Scholer, H. R., Ruppert, S., Suzuki, N., Chowdhury, K. & Gruss, P. New type of POU domain in germ line-specific protein Oct-4. Nature 344, 435-9. (1990).
14. Rosner, M. H. et al. A POU-domain transcription factor in early stem cells and germ cells of the mammalian embryo. Nature 345, 686-92. (1990).
15. Niwa, H., Burdon, T., Chambers, I. & Smith, A. Self-renewal of pluripotent embryonic stem cells is mediated via activation of STAT3. Genes Dev 12, 2048-60. (1998).
16. Ramalho-Santos, M., Yoon, S., Matsuzaki, Y., Mulligan, R. C. & Melton, D. A. “Stemness”: transcriptional profiling of embryonic and adult stem cells. Science 298, 597-600 (2002).
17. Brivanlou, A. H. & Darnell, J. E., Jr. Signal transduction and the control of gene expression. Science 295, 813-8 (2002).
18. van Es, J. H., Barker, N. & Clevers, H. You Wnt some, you lose some: oncogenes in the Wnt signaling pathway. Curr Opin Genet Dev 13, 28-33 (2003).
19. Moon, R. T., Bowerman, B., Boutros, M. & Perrimon, N. The promise and perils of Wnt signaling through beta-catenin. Science 296, 1644-6 (2002).
20. Doble, B. W. & Woodgett, J. R. GSK-3: tricks of the trade for a multi-tasking kinase. J Cell Sci 116, 1175-86 (2003).
21. Korinek, V. et al. Constitutive transcriptional activation by a beta-catenin-Tcf complex in APC−/− colon carcinoma. Science 275, 1784-7 (1997).
22. Tetsu, O. & McCormick, F. Beta-catenin regulates expression of cyclin D1 in colon carcinoma cells. Nature 398, 422-6 (1999).
23. Nagai, T. et al. A variant of yellow fluorescent protein with fast and efficient maturation for cell-biological applications. Nat Biotechnol 20, 87-90 (2002).
24. Hosler, B. A., LaRosa, G. J., Grippo, J. F. & Gudas, L. J. Expression of REX-1, a gene containing zinc finger motifs, is rapidly reduced by retinoic acid in F9 teratocarcinoma cells. Mol Cell Biol 9, 5623-9 (1989).
25. Molenaar, M. et al. XTcf-3 transcription factor mediates beta-catenin-induced axis formation in Xenopus embryos. Cell 86, 391-9 (1996).
26. Vonica, A. & Gumbiner, B. M. Zygotic Wnt activity is required for Brachyury expression in the early Xenopus laevis embryo. Dev Biol 250, 112-27 (2002).
27. Chambers, I. et al. Functional expression cloning of nanog, a pluripotency sustaining factor in embryonic stem cells. Cell 113, 643-55 (2003).
28. Mitsui, K. et al. The Homeoprotein Nanog Is Required for Maintenance of Pluripotency in Mouse Epiblast and ES Cells. Cell 113, 631-42 (2003).
29. Itskovitz-Eldor, J. et al. Differentiation of human embryonic stem cells into embryoid bodies compromising the three embryonic germ layers. Mol Med 6, 88-95. (2000).
30. Kawasaki, H. et al. Generation of dopaminergic neurons and pigmented epithelia from primate ES cells by stromal cell-derived inducing activity. Proc Natl Acad Sci USA 99, 1580-5 (2002).
31. Dani, C. et al. Paracrine induction of stem cell renewal by LIF-deficient cells: a new ES cell regulatory pathway. Dev Biol 203, 149-62. (1998).
32. Niwa, H. Molecular mechanism to maintain stem cell renewal of ES cells. Cell Struct Funct 26, 137-48 (2001).
33. Niwa, H., Miyazaki, J. & Smith, A. G. Quantitative expression of Oct-3/4 defines differentiation, dedifferentiation or self-renewal of ES cells. Nat Genet 24, 372-6. (2000).
34. Huelsken, J. et al. Requirement for beta-catenin in anterior-posterior axis formation in mice. J Cell Biol 148, 567-78 (2000).
35. Conacci-Sorrell, M. E. et al. Nr-CAM is a target gene of the beta-catenin/LEF-1 pathway in melanoma and colon cancer and its expression enhances motility and confers tumorigenesis. Genes Dev 16, 2058-72 (2002).
36. Lloyd, S., Fleming, T. P. & Collins, J. E. Expression of Wnt genes during mouse preimplantation development. Gene Expr Patterns 3, 309-12 (2003).
37. Korinek, V. et al. Depletion of epithelial stem-cell compartments in the small intestine of mice lacking Tcf-4. Nat Genet 19, 379-83 (1998).
38. Kubo, F., Takeichi, M. & Nakagawa, S. Wnt2b controls retinal cell differentiation at the ciliary marginal zone. Development 130, 587-98 (2003).
39. Gat, U., DasGupta, R., Degenstein, L. & Fuchs, E. De Novo hair follicle morphogenesis and hair tumors in mice expressing a truncated beta-catenin in skin. Cell 95, 605-14 (1998).
40. Merrill, B. J., Gat, U., DasGupta, R. & Fuchs, E. Tcf3 and Lef1 regulate lineage differentiation of multipotent stem cells in skin. Genes Dev 15, 1688-705 (2001).
41. Chenn, A. & Walsh, C. A. Regulation of cerebral cortical size by control of cell cycle exit in neural precursors. Science 297, 365-9 (2002).
42. Reya, T. et al. A role for Wnt signalling in self-renewal of haematopoietic stem cells. Nature 423, 409-14 (2003).
43. Giles, R. H., van Es, J. H. & Clevers, H. Caught up in a Wnt storm: Wnt signaling in cancer. Biochim Biophys Acta 1653, 1-24 (2003).
44. Kielman, M. F. et al. Apc modulates embryonic stem-cell differentiation by controlling the dosage of beta-catenin signaling. Nat Genet 32, 594-605 (2002).
45. Aubert, J., Dunstan, H., Chambers, I. & Smith, A. Functional gene screening in embryonic stem cells implicates Wnt antagonism in neural differentiation. Nat Biotechnol 20, 1240-5 (2002).
46. Ding, S. et al. Synthetic small molecules that control stem cell fate. Proc Natl Acad Sci USA (2003).
47. Willert, K. et al. Wnt proteins are lipid-modified and can act as stem cell growth factors. Nature 423, 448-52 (2003).

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

What is claimed:

1. A method of maintaining the undifferentiated state of an embryonic stem cell, said method comprising contacting the stem cell in vitro with a molecule that antagonizes GSK-3 activity such that the cell divides but does not differentiate, wherein said contacting is in the absence of a feeder layer.

2. The method according to claim 1, wherein the molecule is LiCl.

3. The method according to claim 1, wherein the molecule is 6-bromoindirubin-3′-oxime.

4. The method according to claim 1 further comprising the step of removing the molecule from contact with the stem cell.

5. The method according to claim 1, wherein the stem cell is a human stem cell.

6. A method of maintaining the undifferentiated state of an embryonic stem cell, said method comprising contacting said stem cell with 6-bromoindirubin-3′-oxime such that the cells divides but does not differentiate.

7. The method according to claim 6, wherein said contacting is in the absence of a feeder layer.

8. The method according to claim 6, wherein said contacting is in vitro.

9. The method according to claim 6, wherein the stem cell is a human stem cell.

10. An isolated embryonic stem cell in contact with 6-bromoindirubin-3′-oxime.

11. An embryonic stem cell that is the progeny of a second embryonic stem cell that was previously contacted with 6-bromoindirubin-3′-oxime.

12. The embryonic stem cell of claim 11 which is isolated.

13. An embryonic stem cell line produced by the process comprising isolating embryonic stem cells from an embryo and culturing the isolated embryonic stem cells in the presence of a molecule that antagonizes GSK-3 activity such that the isolated embryonic stem cells divide but do not differentiate, wherein said isolating and culturing is in the absence of a feeder layer.

14. The embryonic cell line according to claim 13, wherein said molecule is 6-bromoindirubin-3′-oxime.

15. The embryonic cell line according to claim 13, wherein said molecule is LiCl.

16. The embryonic cell line according to claim 13, wherein the embryonic stem cells are human embryonic stem cells.

17. An embryonic stem cell line produced by the process comprising isolating embryonic stem cells from an embryo and culturing the isolated cells in the presence of 6-bromoindirubin-3′-oxime such that the isolated embryonic stem cells divide but do not differentiate.

18. The embryonic cell line of claim 17, wherein said culturing is in the absence of a feeder layer.

19. The embryonic cell line of claim 17, wherein the embryonic stem cells are human embryonic stem cells.

20. A method of obtaining an embryonic stem cell line comprising isolating embryonic stem cells from an embryo and culturing the isolated embryonic stem cells in the presence of a molecule that antagonizes GSK-3 activity such that the isolated embryonic stem cells divide but do not differentiate, wherein said isolating and culturing is in the absence of a feeder layer.

21. The method according to claim 20, wherein the molecule is LiCl.

22. The method according to claim 20, wherein the molecule is 6-bromoindirubin-3′-oxime.

23. The method according to claim 20, wherein the embryo is a human embryo.

24. A method of obtaining an embryonic stem cell line comprising isolating embryonic stem cells from an embryo and culturing the isolated embryonic stem cells in the presence of 6-bromoindirubin-3′-oxime such that the embryonic stem cells divide but do not differentiate.

25. The method according to claim 24, wherein said isolating and culturing is in the absence of a feeder layer.

26. The method according to claim 24, wherein the embryo is a human embryo.

27. A human embryonic cell line, which cell line has not been in contact with a feeder layer and has not been in contact with an exogenous cell extract.

28. A human embryonic cell line, which cell line has not been in contact with a feeder layer and has not been in contact with a recombinant human protein.

29. The embryonic stem cell line according to claim 13, wherein the embryonic stem cells are isolated and cultured in the absence of exogenous cell extract or serum.

30. The embryonic stem cell line according to claim 13, wherein the embryonic stem cells are recombinant embryonic stem cells.

31. The recombinant embryonic stem cells according to claim 30, which express a prophylactic or therapeutic protein.
